(19) United States
(12) Patent Application Publication
(10) Pub. No.: US 2026/0327698 A1
(43) Pub. Date: Oct. 22, 2026

(54) TWO-COLOR SEQUENCING METHOD AND KIT THEREOF

(71) Applicants: GENEMIND BIOSCIENCES COMPANY LIMITED, Shenzhen (CN); SHENZHEN JUMAI BIOTECHNOLOGY CO., LTD., Shenzhen (CN)

(72) Inventors: Meirong CHEN, Shenzhen (CN); Bin LIU, Shenzhen (CN); Huan JIN, Shenzhen (CN); Fang CHEN, Shenzhen (CN); Lei SUN, Shenzhen (CN)

(73) Assignees: GENEMIND BIOSCIENCES COMPANY LIMITED, Shenzhen (CN); SHENZHEN JUMAI BIOTECHNOLOGY CO., LTD., Shenzhen (CN)

(21) Appl. No.: 19/529,192

(22) Filed: Feb. 4, 2026

(30) Foreign Application Priority Data
Feb. 20, 2025 (CN) 202510195586.X

(51) Int. Cl. C12Q 1/6869; C12Q 1/6823; C12Q 1/6825; G01N 21/64

(52) U.S. Cl. CPC C12Q 1/6869; C12Q 1/6823; C12Q 1/6825; G01N 21/6456

(57) ABSTRACT

The present disclosure relates to the technical field of biological detection, and in particular, to a two-color sequencing method and a kit thereof. Through the optimization of the concentration ratios of the reaction substances and the reaction parameters, the present disclosure develops a two-color sequencing method suitable for a single-molecule sequencing platform. The two-color sequencing method is used for single-molecule sequencing, and is beneficial for reducing error rate, and improving sequencing quality.

### CROSS-REFERENCE TO RELATED APPLICATIONS

This application claims the priority benefit of Chinese application serial No. 202510195586.X filed on Feb. 20, 2025, the entirety of which is hereby incorporated by reference herein and made a part of the specification.

### TECHNICAL FIELD

The present disclosure relates to the technical field of biological detection, and in particular, to a two-color sequencing method and a kit thereof.

### BACKGROUND OF THE INVENTION

Compared with the second-generation sequencing technology, a major characteristic of single-molecule sequencing is that no PCR clustering process is involved. Since signals generated during a clustering-based enrichment sequencing process are not available, the sequencing read length and sequencing quality are subject to limitations. Based on the number of types of nucleotides or nucleotide analogs thereof added in each polymerase chain reaction, the single-molecule sequencing can be classified into one-color sequencing and multi-color sequencing, where common multi-color sequencing includes two-color sequencing and four-color sequencing.

In a typical two-color sequencing approach, four types of reversible terminators with two types of fluorescent labels are mixed in pairs and sequentially added for sequencing, such that two cycles of polymerase chain reactions are required to complete reactions of the four nucleotides. Compared with the one-color sequencing kit, the overall sequencing time can be reduced by approximately one half. How to improve sequencing quality on the premise of two-color sequencing remains an issue of concern.

### SUMMARY

Based on at least the problems described above, the present disclosure provides a two-color sequencing method and a kit thereof, which aim to improve the sequencing quality by optimizing the structural composition of the reversible terminators and the sequencing process.

The two-color sequencing method provided herein includes the following common steps:
causing four types of reversible terminators to undergo a polymerization reaction with a nucleic acid template-primer complex, where the four types of reversible terminators are each independently linked to a nucleotide or a derivative thereof and a fluorophore via a cleavable group, the cleavable groups of two types of the reversible terminators being identical, the fluorophores of two types of the reversible terminators being identical, and the cleavable groups carrying identical fluorophores being different or requiring different cleavage conditions;
exciting the fluorophores to emit fluorescence, and acquiring fluorescence signals to obtain a first image;
cleaving one type of the cleavable groups by a first reaction;
acquiring fluorescence signals of the remaining fluorophores to obtain a second image; and
determining, based on the first image and the second image, types of reversible terminators participating in the polymerization reaction.

As one implementation of the two-color sequencing method of the present application, the four types of reversible terminators are respectively a first reversible terminator, a second reversible terminator, a third reversible terminator, and a fourth reversible terminator, and the four types of reversible terminators each independently include: a nucleotide or a derivative thereof, a fluorophore, and a first cleavable group and/or a second cleavable group linking the nucleotide or the derivative thereof and the fluorophore, the nucleotide or the derivative thereof being selected from adenine or a derivative thereof, thymine or a derivative thereof, cytosine or a derivative thereof, or guanine or a derivative thereof, and the fluorophore being selected from a first fluorophore and a second fluorophore that are distinguishable in color;
image 1 includes a first signal, a second signal, a third signal, and a fourth signal from the first reversible terminator, the second reversible terminator, the third reversible terminator, and the fourth reversible terminator, respectively, the first signal and the second signal being different from the third signal and the fourth signal;
image 2 includes a fifth signal, a sixth signal, a seventh signal, and an eighth signal from the first reversible terminator, the second reversible terminator, the third reversible terminator, and the fourth reversible terminator, respectively, the sixth signal being different from the second signal and the seventh signal being different from the third signal; and
after acquiring the second image or determining the types of reversible terminators participating in the polymerization reaction, the other type of the cleavable groups is cleaved by a second reaction.

As one implementation of the two-color sequencing method of the present application, cleavability of the second cleavable group is stronger than cleavability of the first cleavable group, and the first reaction cleaves the second cleavable group, and
the second reaction cleaves the first cleavable group.

As one implementation of the two-color sequencing method of the present application, the two-color sequencing method may include the following steps:
providing four types of reversible terminators: a first reversible terminator, a second reversible terminator, a third reversible terminator, and a fourth reversible terminator, where the four types of reversible terminators each independently include: a nucleotide or a derivative thereof, a fluorophore, and a first cleavable group and/or a second cleavable group linking the nucleotide or the derivative thereof and the fluorophore;
the nucleotide or the derivative thereof being selected from adenine or a derivative thereof, thymine or a derivative thereof, cytosine or a derivative thereof, or guanine or a derivative thereof, and the fluorophore being selected from a first fluorophore and a second fluorophore that are distinguishable in color;
the reversible terminators being bound to a nucleic acid template-primer complex by a polymerase chain reaction, and the primer being extended to obtain an extension product;
exciting and acquiring fluorescence signals of the extension product to obtain image 1, where image 1 includes a first signal, a second signal, a third signal, and a fourth signal from the first reversible terminator, the second reversible terminator, the third reversible terminator, and the fourth reversible terminator, respectively, and the first signal and the second signal are different from the third signal and the fourth signal;
after obtaining image 1, subjecting the extension product to a first reaction to obtain a reaction product;
acquiring fluorescence signals of the reaction product to obtain image 2, where image 2 includes a fifth signal, a sixth signal, a seventh signal, and an eighth signal from the first reversible terminator, the second reversible terminator, the third reversible terminator, and the fourth reversible terminator, respectively, and the sixth signal is different from the second signal and the seventh signal is different from the third signal;
determining, based on optical signals of images 1 and 2, types of reversible terminators bound to the nucleic acid template-primer complex; and
subjecting the reaction product to a second reaction,
where the first reaction cleaves the second cleavable group; and
the second reaction cleaves the first cleavable group.

Based on the above embodiments, as one implementation of the two-color sequencing method of the present application, the cleavability of the second cleavable group is stronger than the cleavability of the first cleavable group.

As one implementation of the two-color sequencing method of the present application, the first cleavable group is selected from a group including at least one of —N3-, —CH₂F, —C(O)NHBu, —CHF₂, —C(O)OH, —C₆, and/or —ArC₆.

Based on the above embodiments, as one implementation of the two-color sequencing method of the present application, the first cleavable group is selected from at least one of —N3-, —CH₂F, —C(O)NHBu, —CHF₂, —C(O)OH, —C₆, and/or —ArC₆.

Based on the above embodiments, as one implementation of the two-color sequencing method of the present application, the first cleavable group is —N3-.

Based on the above embodiments, as one implementation of the two-color sequencing method of the present application, the second cleavable group is selected from a group including a disulfide bond. As one implementation of the two-color sequencing method of the present application, the second cleavable group is a disulfide bond.

Based on the above embodiments, as one implementation of the two-color sequencing method of the present application, the first fluorophore and the second fluorophore are each independently selected from one of or a combination of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin.

Based on the above embodiments, as one implementation of the two-color sequencing method of the present application, a color combination of the first fluorophore and the second fluorophore is one of the following combinations: the fluorescence color of the first fluorophore being red, and the fluorescence color of the second fluorophore being green;
the fluorescence color of the first fluorophore being red, and the fluorescence color of the second fluorophore being blue;
the fluorescence color of the first fluorophore being green, and the fluorescence color of the second fluorophore being red;
the fluorescence color of the first fluorophore being green, and the fluorescence color of the second fluorophore being blue;
the fluorescence color of the first fluorophore being blue, and the fluorescence color of the second fluorophore being red; and
the fluorescence color of the first fluorophore being blue, and the fluorescence color of the second fluorophore being green. Based on the above embodiments, as one implementation of the two-color sequencing method of the present application, the combination of the first fluorescence signal and the second fluorescence signal is a combination formed by red and green fluorescence signals; or the combination of the first fluorescence signal and the second fluorescence signal is a combination formed by red and blue fluorescence signals.

Based on the above embodiments, as one implementation of the two-color sequencing method of the present application, the fluorophore having a fluorescence color of green includes Atto532, Alexa Fluor532, Atto Rho6G, or CY3;
the fluorophore having a fluorescence color of red includes: Atto647N, CY5, GM656, or Alexa Fluor647; and
the fluorophore having a fluorescence color of blue includes: Atto390, Alexa Fluor405, or coumarin.

Based on the above embodiments, as one implementation of the two-color sequencing method of the present application, the first signal is the same as the second signal, and the third signal is the same as the fourth signal.

Based on the above embodiments, as one implementation of the two-color sequencing method of the present application, any two types of the reversible terminators have the same cleavable group.

Based on the above embodiments, as one implementation of the two-color sequencing method of the present application, the fluorophores of the reversible terminators having the same cleavable group have different fluorescence colors.

In combination with any one or more of the above cases, as one implementation of the sequencing method of the present application, each type of the reversible terminator has only one type of cleavable group.

As one implementation of the two-color sequencing method of the present application, the reversible terminators have the following structures:
the structure of the first reversible terminator being: a first nucleotide or a derivative thereof-the first cleavable group-the first fluorophore;
the structure of the second reversible terminator being: a second nucleotide or a derivative thereof-the second cleavable group-the first fluorophore;
the structure of the third reversible terminator being: a third nucleotide or a derivative thereof-the second cleavable group-the second fluorophore; and
the structure of the fourth reversible terminator being: a fourth nucleotide or a derivative thereof-the first cleavable group-the second fluorophore,
where the first nucleotide or the derivative thereof, the second nucleotide or the derivative thereof, the third nucleotide or the derivative thereof, and the fourth nucleotide or the derivative thereof are independently selected from one of adenine or a derivative thereof, thymine or a derivative thereof, cytosine guanine or a derivative thereof, and guanine or a derivative thereof, and the first nucleotide or the derivative thereof, the second nucleotide or the derivative thereof, the third nucleotide or the derivative thereof, and the fourth nucleotide or the derivative thereof are different from one another. For convenience of description, the two-color sequencing method corresponding to this implementation is named as two-color sequencing method 1.

As one implementation of the two-color sequencing method of the present application, the four types of reversible terminators in two-color sequencing method 1 include at least one of the following combinations 1-16; or the four types of reversible terminators include at least one of the following combinations 17-32:
combination 1: adenine or a derivative thereof-N3-Atto647N; thymine or a derivative thereof-SS-Atto532; cytosine or a derivative thereof-N3-Alexa Fluor532; and guanine or a derivative thereof-SS-CY5;
combination 2: adenine or a derivative thereof-N3-Atto647N; thymine or a derivative thereof-SS-Atto532; cytosine or a derivative thereof-N3-Alexa Fluor532; and guanine or a derivative thereof-SS-Atto647N;
combination 3: adenine or a derivative thereof-N3-Atto647N; thymine or a derivative thereof-SS-Alexa Fluor532; cytosine or a derivative thereof-N3-Atto532; and guanine or a derivative thereof-SS-Atto647N;
combination 4: adenine or a derivative thereof-N3-CY5; thymine or a derivative thereof-SS-Atto532; cytosine or a derivative thereof-N3-Alexa Fluor532; and guanine or a derivative thereof-SS-CY5;
combination 5: adenine or a derivative thereof-N3-CY5; thymine or a derivative thereof-SS-Alexa Fluor532; cytosine or a derivative thereof-N3-Atto532; and guanine or a derivative thereof-SS-CY5;
combination 6: adenine or a derivative thereof-N3-CY5; thymine or a derivative thereof-SS-Atto532; cytosine or a derivative thereof-N3-Atto532; and guanine or a derivative thereof-SS-CY5;
combination 7: adenine or a derivative thereof-N3-CY5; thymine or a derivative thereof-SS-Alexa Fluor532; cytosine or a derivative thereof-N3-Alexa Fluor532; and guanine or a derivative thereof-SS-CY5;
combination 8: adenine or a derivative thereof-N3-Atto647N; thymine or a derivative thereof-SS-Atto532; cytosine or a derivative thereof-N3-Atto532; and guanine or a derivative thereof-SS-CY5;
combination 9: adenine or a derivative thereof-N3-Atto647N; thymine or a derivative thereof-SS-Alexa Fluor532; cytosine or a derivative thereof-N3-Atto532; and guanine or a derivative thereof-SS-CY5;
combination 10: adenine or a derivative thereof-N3-Atto647N; thymine or a derivative thereof-SS-Alexa Fluor532; cytosine or a derivative thereof-N3-Alexa Fluor532; and guanine or a derivative thereof-SS-CY5;
combination 11: adenine or a derivative thereof-N3-CY5; thymine or a derivative thereof-SS-Alexa Fluor532; cytosine or a derivative thereof-N3-Alexa Fluor532; and guanine or a derivative thereof-SS-Atto647N;
combination 12: adenine or a derivative thereof-N3-CY5; thymine or a derivative thereof-SS-Atto532; cytosine or a derivative thereof-N3-Atto532; and guanine or a derivative thereof-SS-Atto647N;
combination 13: adenine or a derivative thereof-N3-CY5; thymine or a derivative thereof-SS-Atto532; cytosine or a derivative thereof-N3-Alexa Fluor532; and guanine or a derivative thereof-SS-Atto647N;
combination 14: adenine or a derivative thereof-N3-CY5; thymine or a derivative thereof-SS-Alexa Fluor532; cytosine or a derivative thereof-N3-Atto532; and guanine or a derivative thereof-SS-Atto647N;
combination 15: adenine or a derivative thereof-N3-Atto647N; thymine or a derivative thereof-SS-Alexa Fluor532; cytosine or a derivative thereof-N3-Alexa Fluor532; and guanine or a derivative thereof-SS-Atto647N;
combination 16: adenine or a derivative thereof-N3-Atto647N; thymine or a derivative thereof-SS-Atto532; cytosine or a derivative thereof-N3-Atto532; and guanine or a derivative thereof-SS-Atto647N;
combination 17: adenine or a derivative thereof-N3-Atto532; thymine or a derivative thereof-SS-Atto647N; cytosine or a derivative thereof-N3-Atto647N; and guanine or a derivative thereof-SS-Atto532;
combination 18: adenine or a derivative thereof-N3-Alexa Fluor532; thymine or a derivative thereof-SS-Atto647N; cytosine or a derivative thereof-N3-Atto647N; and guanine or a derivative thereof-SS-Atto532;
combination 19: adenine or a derivative thereof-N3-Atto532; thymine or a derivative thereof-SS-Atto647N; cytosine or a derivative thereof-N3-Atto647N; and guanine or a derivative thereof-SS-Alexa Fluor532;
combination 20: adenine or a derivative thereof-N3-Alexa Fluor532; thymine or a derivative thereof-SS-Atto647N; cytosine or a derivative thereof-N3-Atto647N; and guanine or a derivative thereof-SS-Alexa Fluor532;
combination 21: adenine or a derivative thereof-N3-Atto532; thymine or a derivative thereof-SS-CY5; cytosine or a derivative thereof-N3-Atto647N; and guanine or a derivative thereof-SS-Atto532;
combination 22: adenine or a derivative thereof-N3-Alexa Fluor532; thymine or a derivative thereof-SS-CY5; cytosine or a derivative thereof-N3-Atto647N; and guanine or a derivative thereof-SS-Atto532;
combination 23: adenine or a derivative thereof-N3-Atto532; thymine or a derivative thereof-SS-CY5; cytosine or a derivative thereof-N3-Atto647N; and guanine or a derivative thereof-SS-Alexa Fluor532;
combination 24: adenine or a derivative thereof-N3-Alexa Fluor532; thymine or a derivative thereof-SS-CY5; cytosine or a derivative thereof-N3-Atto647N; and guanine or a derivative thereof-SS-Alexa Fluor532;
combination 25: adenine or a derivative thereof-N3-Atto532; thymine or a derivative thereof-SS-Atto647N; cytosine or a derivative thereof-N3-CY5; and guanine or a derivative thereof-SS-Atto532;
combination 26: adenine or a derivative thereof-N3-Alexa Fluor532; thymine or a derivative thereof-SS-Atto647N; cytosine or a derivative thereof-N3-CY5; and guanine or a derivative thereof-SS-Atto532;
combination 27: adenine or a derivative thereof-N3-Atto532; thymine or a derivative thereof-SS-Atto647N; cytosine or a derivative thereof-N3-CY5; and guanine or a derivative thereof-SS-Alexa Fluor532;
combination 28: adenine or a derivative thereof-N3-Alexa Fluor532; thymine or a derivative thereof-SS-Atto647N; cytosine or a derivative thereof-N3-CY5; and guanine or a derivative thereof-SS-Alexa Fluor532;
combination 29: adenine or a derivative thereof-N3-Alexa Fluor532; thymine or a derivative thereof-SS-CY5; cytosine or a derivative thereof-N3-CY5; and guanine or a derivative thereof-SS-Atto532;
combination 30: adenine or a derivative thereof-N3-Atto532; thymine or a derivative thereof-SS-CY5; cytosine or a derivative thereof-N3-CY5; and guanine or a derivative thereof-SS-Alexa Fluor532;
combination 31: adenine or a derivative thereof-N3-Atto532; thymine or a derivative thereof-SS-CY5; cytosine or a derivative thereof-N3-CY5; and guanine or a derivative thereof-SS-Atto532; and
combination 32: adenine or a derivative thereof-N3-Alexa Fluor532; thymine or a derivative thereof-SS-CY5; cytosine or a derivative thereof-N3-CY5; and guanine or a derivative thereof-SS-Alexa Fluor532.

As one possible implementation of the two-color sequencing method of the present application, cleavable groups in combinations 1-32 may be replaced;
in combinations 1-32, —N3- linked to adenine or a derivative thereof is replaced with —SS—, —N3- linked to cytosine or a derivative thereof-N3 is replaced with —SS—, —SS— linked to thymine or a derivative thereof is replaced with —N3-, and —SS— linked to guanine or a derivative thereof is replaced with —N3; or
in combinations 1-32, —N3- linked to adenine or a derivative thereof is replaced with —SS—, and —SS— linked to guanine or a derivative thereof is replaced with —N3-; or
in combinations 1-32, —SS— linked to thymine or a derivative thereof is replaced with —N3-, and —N3- linked to cytosine or a derivative thereof is replaced with —SS—.

As one possible implementation of the two-color sequencing method of the present application,
in combinations 1-32 of the above cases or after replacing the cleavable groups in combinations 1-32, CY5 and/or Atto647N is replaced with GM656 or Alexa Fluor647; and/or Atto532 and/or Alexa Fluor532 is replaced with Atto Rho6G or CY3.

As one possible implementation of the two-color sequencing method of the present application, in combinations 1-32 of the above cases or after replacing the cleavable groups in combinations 1-32, CY5 and/or Atto647N is replaced with one type of blue-emitting fluorescent dyes.

Specifically, the nucleotide or the derivative thereof includes a deoxyribonucleotide or a derivative thereof, or a ribonucleotide or a derivative thereof; in a specific embodiment of the present disclosure, the nucleotide or the derivative thereof is a deoxyribonucleotide or a derivative thereof, where adenine deoxyribonucleotide or the derivative thereof is represented by dA, guanine deoxyribonucleotide or the derivative thereof is represented by dG, cytosine deoxyribonucleotide or the derivative thereof is represented by dC, and thymine deoxyribonucleotide or the derivative thereof is represented by dT. In this case, the types of reversible terminators and the detection procedures compatible with two-color sequencing method 1 are shown in Table 1:

TABLE 1
Types of reversible terminators and detection procedures
compatible with two-color sequencing method 1

| Reversible terminator | Image 1 | First reaction | Image 2 | Second reaction |
|---|---|---|---|---|
| dA-N3-Atto647N red | 1 first signal | No cleaving | 1 fifth signal | Cleaving N3 |
| dG-SS-Atto647N red | 1 second signal | Cleaving SS | 0 sixth signal | / |
| dT-SS-Atto532 green | 1 third signal | Cleaving SS | 0 seventh signal | / |
| dC-N3-Atto532 green | 1 fourth signal | No cleaving | 1 eighth signal | Cleaving N3 |

Note:
1 represents a signal, and 0 represents no signal; SS represents the second cleavable group, and N3 represents the first cleavable group.

As another implementation of the sequencing method of the present application, any two types of the reversible terminators have one type of cleavable group, and the other two types of the reversible terminators have two types of cleavable groups.

As one possible implementation of the two-color sequencing method of the present application, the reversible terminators have the following structures:
the structure of the first reversible terminator being: a first nucleotide or a derivative thereof-the first cleavable group-the first fluorophore;
the structure of the second reversible terminator being: a second nucleotide or a derivative thereof-the first cleavable group-the second cleavable group-the first fluorophore;
the structure of the third reversible terminator being: a third nucleotide or a derivative thereof-the first cleavable group-the second cleavable group-the second fluorophore;
the structure of the fourth reversible terminator being: a fourth nucleotide or a derivative thereof-the first cleavable group-the second fluorophore,
where the first nucleotide or the derivative thereof, the second nucleotide or the derivative thereof, the third nucleotide or the derivative thereof, and the fourth nucleotide or the derivative thereof are independently selected from one of adenine or a derivative thereof, thymine or a derivative thereof, cytosine guanine or a derivative thereof, and guanine or a derivative thereof, and the first nucleotide or the derivative thereof, the second nucleotide or the derivative thereof, the third nucleotide or the derivative thereof, and the fourth nucleotide or the derivative thereof are different from one another.

For convenience of description, the two-color sequencing method corresponding to this implementation is named as two-color sequencing method 2.

As one implementation of the two-color sequencing method of the present application, the four types of reversible terminators in two-color sequencing method 2 include at least one of the following combinations 33-48; or the four types of reversible terminators include at least one of the following combinations 49-64:
combination 33: adenine or a derivative thereof-N3-Atto647N; thymine or a derivative thereof-N3-SS-Atto532; cytosine or a derivative thereof-N3-Alexa Fluor532; and guanine or a derivative thereof-N3-SS-CY5;
combination 34: adenine or a derivative thereof-N3-Atto647N; thymine or a derivative thereof-N3-SS-Atto532; cytosine or a derivative thereof-N3-Alexa Fluor532; and guanine or a derivative thereof-N3-SS-Atto647N;
combination 35: adenine or a derivative thereof-N3-Atto647N; thymine or a derivative thereof-N3-SS-Alexa Fluor532; cytosine or a derivative thereof-N3-Atto532; and guanine or a derivative thereof-N3-SS-Atto647N;
combination 36: adenine or a derivative thereof-N3-CY5; thymine or a derivative thereof-N3-SS-Atto532; cytosine or a derivative thereof-N3-Alexa Fluor532; and guanine or a derivative thereof-N3-SS-CY5;
combination 37: adenine or a derivative thereof-N3-CY5; thymine or a derivative thereof-N3-SS-Alexa Fluor532; cytosine or a derivative thereof-N3-Atto532; and guanine or a derivative thereof-N3-SS-CY5;
combination 38: adenine or a derivative thereof-N3-CY5; thymine or a derivative thereof-N3-SS-Atto532; cytosine or a derivative thereof-N3-Atto532; and guanine or a derivative thereof-N3-SS-CY5;
combination 39: adenine or a derivative thereof-N3-CY5; thymine or a derivative thereof-N3-SS-Alexa Fluor532; cytosine or a derivative thereof-N3-Alexa Fluor532; and guanine or a derivative thereof-N3-SS-CY5;
combination 40: adenine or a derivative thereof-N3-Atto647N; thymine or a derivative thereof-N3-SS-Atto532; cytosine or a derivative thereof-N3-Atto532; and guanine or a derivative thereof-N3-SS-CY5;
combination 41: adenine or a derivative thereof-N3-Atto647N; thymine or a derivative thereof-N3-SS-Alexa Fluor532; cytosine or a derivative thereof-N3-Atto532; and guanine or a derivative thereof-N3-SS-CY5;
combination 42: adenine or a derivative thereof-N3-Atto647N; thymine or a derivative thereof-N3-SS-Alexa Fluor532; cytosine or a derivative thereof-N3-Alexa Fluor532; and guanine or a derivative thereof-N3-SS-CY5;
combination 43: adenine or a derivative thereof-N3-CY5; thymine or a derivative thereof-N3-SS-Alexa Fluor532; cytosine or a derivative thereof-N3-Alexa Fluor532; and guanine or a derivative thereof-N3-SS-Atto647N;
combination 44: adenine or a derivative thereof-N3-CY5; thymine or a derivative thereof-N3-SS-Atto532; cytosine or a derivative thereof-N3-Atto532; and guanine or a derivative thereof-N3-SS-Atto647N;
combination 45: adenine or a derivative thereof-N3-CY5; thymine or a derivative thereof-N3-SS-Atto532; cytosine or a derivative thereof-N3-Alexa Fluor532; and guanine or a derivative thereof-N3-SS-Atto647N;
combination 46: adenine or a derivative thereof-N3-CY5; thymine or a derivative thereof-N3-SS-Alexa Fluor532; cytosine or a derivative thereof-N3-Atto532; and guanine or a derivative thereof-N3-SS-Atto647N;
combination 47: adenine or a derivative thereof-N3-Atto647N; thymine or a derivative thereof-N3-SS-Alexa Fluor532; cytosine or a derivative thereof-N3-Alexa Fluor532; and guanine or a derivative thereof-N3-SS-Atto647N;
combination 48: adenine or a derivative thereof-N3-Atto647N; thymine or a derivative thereof-N3-SS-Atto532; cytosine or a derivative thereof-N3-Atto532; and guanine or a derivative thereof-N3-SS-Atto647N;
combination 49: adenine or a derivative thereof-N3-Atto532; thymine or a derivative thereof-N3-SS-Atto647N; cytosine or a derivative thereof-N3-Atto647N; and guanine or a derivative thereof-N3-SS-Atto532;
combination 50: adenine or a derivative thereof-N3-Alexa Fluor532; thymine or a derivative thereof-N3-SS-Atto647N; cytosine or a derivative thereof-N3-Atto647N; and guanine or a derivative thereof-N3-SS-Atto532;
combination 51: adenine or a derivative thereof-N3-Atto532; thymine or a derivative thereof-N3-SS-Atto647N; cytosine or a derivative thereof-N3-Atto647N; and guanine or a derivative thereof-N3-SS-Alexa Fluor532;
combination 52: adenine or a derivative thereof-N3-Alexa Fluor532; thymine or a derivative thereof-N3-SS-Atto647N; cytosine or a derivative thereof-N3-Atto647N; and guanine or a derivative thereof-N3-SS-Alexa Fluor532;
combination 53: adenine or a derivative thereof-N3-Atto532; thymine or a derivative thereof-N3-SS-CY5; cytosine or a derivative thereof-N3-Atto647N; and guanine or a derivative thereof-N3-SS-Atto532;
combination 54: adenine or a derivative thereof-N3-Alexa Fluor532; thymine or a derivative thereof-N3-SS-CY5; cytosine or a derivative thereof-N3-Atto647N; and guanine or a derivative thereof-N3-SS-Atto532;
combination 55: adenine or a derivative thereof-N3-Atto532; thymine or a derivative thereof-N3-SS-CY5; cytosine or a derivative thereof-N3-Atto647N; and guanine or a derivative thereof-N3-SS-Alexa Fluor532;
combination 56: adenine or a derivative thereof-N3-Alexa Fluor532; thymine or a derivative thereof-N3-SS-CY5; cytosine or a derivative thereof-N3-Atto647N; and guanine or a derivative thereof-N3-SS-Alexa Fluor532;
combination 57: adenine or a derivative thereof-N3-Atto532; thymine or a derivative thereof-N3-SS-Atto647N; cytosine or a derivative thereof-N3-CY5; and guanine or a derivative thereof-N3-SS-Atto532;
combination 58: adenine or a derivative thereof-N3-Alexa Fluor532; thymine or a derivative thereof-N3-SS-Atto647N; cytosine or a derivative thereof-N3-CY5; and guanine or a derivative thereof-N3-SS-Atto532;
combination 59: adenine or a derivative thereof-N3-Atto532; thymine or a derivative thereof-N3-SS-Atto647N; cytosine or a derivative thereof-N3-CY5; and guanine or a derivative thereof-N3-SS-Alexa Fluor532;
combination 60: adenine or a derivative thereof-N3-Alexa Fluor532; thymine or a derivative thereof-N3-SS-Atto647N; cytosine or a derivative thereof-N3-CY5; and guanine or a derivative thereof-N3-SS-Alexa Fluor532;
combination 61: adenine or a derivative thereof-N3-Alexa Fluor532; thymine or a derivative thereof-N3-SS-CY5; cytosine or a derivative thereof-N3-CY5; and guanine or a derivative thereof-N3-SS-Atto532;
combination 62: adenine or a derivative thereof-N3-Atto532; thymine or a derivative thereof-N3-SS-CY5; cytosine or a derivative thereof-N3-CY5; and guanine or a derivative thereof-N3-SS-Alexa Fluor532;
combination 63: adenine or a derivative thereof-N3-Atto532; thymine or a derivative thereof-N3-SS-CY5; cytosine or a derivative thereof-N3-CY5; and guanine or a derivative thereof-N3-SS-Atto532; and
combination 64: adenine or a derivative thereof-N3-Alexa Fluor532; and thymine or a derivative thereof-N3-SS-CY5.

As one possible implementation of the two-color sequencing method of the present application, cleavable groups in combinations 33-64 may be replaced;
in combinations 33-64, a single cleavable group-N3- linked to adenine or a derivative thereof is replaced with a double cleavable group-N3-SS—, a single cleavable group-N3- linked to cytosine or a derivative thereof is replaced with —N3-SS—, a double cleavable group-N3-SS-linked to thymine or a derivative thereof is replaced with a single cleavable group-N3-, and a double cleavable group-N3-SS— linked to guanine or a derivative thereof is replaced with a single cleavable group-N3-; or
in combinations 33-64, a single cleavable group-N3- linked to adenine or a derivative thereof is replaced with a double cleavable group-N3-SS—, and a double cleavable group-N3-SS— linked to guanine or a derivative thereof is replaced with a single cleavable group-N3-; or
in combinations 33-64, a double cleavable group-N3-SS— linked to thymine or a derivative thereof is replaced with a single cleavable group-N3-, and a single cleavable group-N3- linked to cytosine or a derivative thereof is replaced with a double cleavable group-N3-SS—.

As one possible implementation of the two-color sequencing method of the present application, in combinations 33-64 of the above cases or after replacing the cleavable groups in combinations 33-64, CY5 and/or Atto647N is replaced with GM656 or Alexa Fluor647; and/or Atto532 and/or Alexa Fluor532 is replaced with Atto Rho6G or CY3, or
as one possible implementation of the two-color sequencing method of the present application, in combinations 33-64 of the above cases or after replacing the cleavable groups in combinations 33-64, CY5 and/or Atto647N is replaced with one type of blue-emitting fluorescent dyes.

Specifically, the nucleotide or the derivative thereof includes a deoxyribonucleotide or a derivative thereof, or a ribonucleotide or a derivative thereof; in a specific embodiment of the present disclosure, the nucleotide or the derivative thereof is a deoxyribonucleotide or a derivative thereof, where adenine deoxyribonucleotide or the derivative thereof is represented by dA, guanine deoxyribonucleotide or the derivative thereof is represented by dG, cytosine deoxyribonucleotide or the derivative thereof is represented by dC, and thymine deoxyribonucleotide or the derivative thereof is represented by dT. As one example, the types of reversible terminators and the detection procedures compatible with two-color sequencing method 2 are shown in Table 2:

TABLE 2
Types of reversible terminators and detection procedures
compatible with two-color sequencing method 2

| Reversible terminator | Image 1 | First reaction | Image 2 | Second reaction |
|---|---|---|---|---|
| dA-N3-Atto647N red | 1 first signal | No cleaving | 1 fifth signal | Cleaving N3 |
| dG-N3-SS-CY5 red | 1 second signal | Cleaving SS | 0 sixth signal | Cleaving N3 |
| dT-N3-SS-Atto532 green | 1 third signal | Cleaving SS | 0 seventh signal | Cleaving N3 |
| dC-N3-Alexa Fluor532 green | 1 fourth signal | No cleaving | 1 eighth signal | Cleaving N3 |

Note:
1 represents a signal, and 0 represents no signal; SS represents the second cleavable group, and N3 represents the first cleavable group.

As one possible implementation of the two-color sequencing method of the present application, the reversible terminators are combination 1.

As one possible implementation of the two-color sequencing method of the present application, in combination 1, the working concentrations of the reversible terminators are: adenine or the derivative thereof-N3-Atto647N at 10 nmol/L-700 nmol/L; thymine or the derivative thereof-N3-SS-Atto532 at 5 nmol/L-60 nmol/L; cytosine or the derivative thereof-N3-Alexa Fluor532 at 10 nmol/L-700 nmol/L; and guanine or the derivative thereof-N3-SS-CY5 at 0.05 nmol/L-5 nmol/L.

In combination with one or a combination of the above various implementations, as one possible implementation of the two-color sequencing method of the present application, a cleavage reagent for cleaving the first cleavable group is an organic phosphine cleavage reagent.

As one possible implementation of the two-color sequencing method of the present application, in the organic phosphine cleavage reagent, the organic phosphine is selected from any one or more of tris(3-hydroxypropyl)phosphine, tris(2-carboxyethyl)phosphine, tris(hydroxymethyl) phosphine, triphenylphosphine, or tris(2-hydroxyethyl)phosphine.

As one possible implementation of the two-color sequencing method of the present application, in the organic phosphine cleavage reagent, the organic phosphine is tris(3-hydroxypropyl)phosphine (THPP).

As one possible implementation of the two-color sequencing method of the present application, the working concentration of the tris(3-hydroxypropyl)phosphine is 10 mmol/L-100 mmol/L.

As one possible implementation of the two-color sequencing method of the present application, the organic phosphine cleavage reagent includes 0.2 mol/L-2 mol/L Tris-Base, 0.2 mol/L-2 mol/L sodium chloride, 10 mmol/L-100 mmol/L THPP, and 0.01%-0.1% Tween 20, at a pH of 8-10.

In combination with one or a combination of the above various implementations, as one possible implementation of the two-color sequencing method of the present application, a cleavage reagent for cleaving the second cleavable group is a reducing agent.

As one possible implementation of the two-color sequencing method of the present application, the reducing agent is selected from any one or more of DTT, glutathione, and β-mercaptoethanol.

As one possible implementation of the two-color sequencing method of the present application, the working concentration of the glutathione is 10 mmol/L-100 mmol/L.

As one possible implementation of the two-color sequencing method of the present application, the reducing agent cleavage reagent includes: 0.2 mol/L-2 mol/L Tris-Base, 0.2 mol/L-2 mol/L sodium chloride, 10 mmol/L-100 mmol/L GSH, and 0.01%-0.1% Tween 20, at a pH of 8.0-10.

As one possible implementation of the two-color sequencing method of the present application, the exposure conditions are: exposure for 100 ms-400 ms under a first wavelength condition, and exposure for 100 ms-400 ms under a second wavelength condition, illustratively, exposure for 60 ms-100 ms at 532 nm and exposure for 60 ms-100 ms at 640 nm.

In the polymerase chain reaction, the polymerase includes one or more of 9°N enzyme, Pfu, KOD1, and mmol/LS2;
specifically, the polymerase is 9°N enzyme, and its working concentration is 0.01 mg/mL-0.1 mg/mL, where specifically, the effect is optimal at a concentration of 0.01 mg/mL; and
a condition of the polymerase chain reaction is: pH 8.0-9.0.

According to the two-color sequencing method provided by the present application, the types of the fluorophores and the cleavable groups of the four types of reversible terminators are set and reasonably matched, such that the four types of reversible terminators can be completely cleaved through two cleavage reactions while simultaneously achieving identification of the four types of reversible terminators incorporated in the sequencing process. In addition, by optimizing the concentrations of the reversible terminators, the concentrations of the first cleavable reagent and the second cleavable reagent, the exposure time and the wavelength, and the like, a plurality of two-color sequencing methods under different conditions can be obtained.

Through the optimization of the concentration ratios of the reaction substances and the reaction parameters, the present disclosure develops a two-color sequencing method suitable for a single-molecule sequencing platform. The two-color sequencing method is used for single-molecule sequencing, and is beneficial for reducing error rate, improving sequencing quality, and shortening sequencing time.

The present application further provides a kit including four types of reversible terminators, cleavage reagent 1, and/or cleavage reagent 2,
where the four types of reversible terminators are independently: a first reversible terminator, a second reversible terminator, a third reversible terminator, and a fourth reversible terminator; the four types of reversible terminators each independently include: a nucleotide or a derivative thereof, a fluorophore, and a first cleavable group and/or a second cleavable group linking the nucleotide or the derivative thereof and the fluorophore; and the first cleavable group and the second cleavable group have different degrees of cleavability under the same cleavable condition.

As one possible implementation of the kit of the present application, the first cleavable group is selected from a group including at least one of —N3-, —CH₂F, —C(O)NHBu, —CHF₂, —C(O)OH, —C₆, and/or —ArC₆.

Based on the above embodiments, as one possible implementation of the kit of the present application, the first cleavable group is selected from at least one of —N3-, —CH₂F, —C(O)NHBu, —CHF₂, —C(O)OH, —C₆, and/or —ArC₆.

Based on the above embodiments, as one possible implementation of the kit of the present application, the first cleavable group is —N3-; and
the second cleavable group is selected from a group including a disulfide bond.

Based on the above embodiments, as one possible implementation of the kit of the present application, the fluorophore label is selected from: any two or more of Atto532, Atto647N, Alexa Fluor532, Alexa Fluor647, Atto Rho6G, Alexa Fluor405, Atto390, CY5, IF700, ROX, CY3, GM656, CY5 rhodamine dye, cyanine dye, coumarin dye, or phycoerythrin.

Based on the above embodiments, as one possible implementation of the kit of the present application, the fluorescence color of the first fluorophore is red, green, or blue; and the fluorescence color of the second fluorophore is red, green, or blue.

Based on the above embodiments, as one possible implementation of the kit of the present application, the combination of the first fluorescence signal and the second fluorescence signal is a combination formed by red and green fluorescence signals; or the combination of the first fluorescence signal and the second fluorescence signal is a combination formed by red and blue fluorescence signals.

Based on the above embodiments, as one possible implementation of the kit of the present application, the fluorophore having a fluorescence color of green includes Atto532, Alexa Fluor532, Atto Rho6G, or CY3;
the fluorophore having a fluorescence color of red includes: Atto647N, CY5, GM656, or Alexa Fluor647;
the fluorophore having a fluorescence color of blue includes: Atto390, Alexa Fluor405, or coumarin; and
any one type of the reversible terminators has any one or two of the first cleavable group and/or the second cleavable group.

Based on the above embodiments, as one possible implementation of the kit of the present application, any two types of the reversible terminators have the same cleavable group.

Based on the above embodiments, as one possible implementation of the kit of the present application, the fluorophores of the reversible terminators having the same cleavable group have different fluorescence colors.

Based on the above embodiments, as one possible implementation of the kit of the present application, the reversible terminators have the following structures:
the structure of the first reversible terminator being: a first nucleotide or a derivative thereof-the first cleavable group-the first fluorophore;
the structure of the second reversible terminator being: a second nucleotide or a derivative thereof-the second cleavable group-the first fluorophore;
the structure of the third reversible terminator being: a third nucleotide or a derivative thereof-the second cleavable group-the second fluorophore; and
the structure of the fourth reversible terminator being: a fourth nucleotide or a derivative thereof-the first cleavable group-the second fluorophore.

Based on the above embodiments, as one possible implementation of the kit of the present application, the reversible terminators have the following structures:
the structure of the first reversible terminator being: a first nucleotide or a derivative thereof-the first cleavable group-the first fluorophore;
the structure of the second reversible terminator being: a second nucleotide or a derivative thereof-the first cleavable group-the second cleavable group-the first fluorophore;
the structure of the third reversible terminator being: a third nucleotide or a derivative thereof-the first cleavable group-the second cleavable group-the second fluorophore;
the structure of the fourth reversible terminator being: a fourth nucleotide or a derivative thereof-the first cleavable group-the second fluorophore,
where the first nucleotide or the derivative thereof, the second nucleotide or the derivative thereof, the third nucleotide or the derivative thereof, and the fourth nucleotide or the derivative thereof are independently selected from one of adenine or a derivative thereof, thymine or a derivative thereof, cytosine guanine or a derivative thereof, and guanine or a derivative thereof, and the first nucleotide or the derivative thereof, the second nucleotide or the derivative thereof, the third nucleotide or the derivative thereof, and the fourth nucleotide or the derivative thereof are different from one another.

Based on the above embodiments, as one possible implementation of the kit of the present application, the cleavage reagent for cleaving the first cleavable group is an organic phosphine cleavage reagent.

Based on the above embodiments, as one possible implementation of the kit of the present application, the cleavage reagent for cleaving the second cleavable group is a reducing agent.

As one possible implementation of the kit of the present application, the reversible terminators include at least one of the following combinations 1-16, or at least one of the combinations 17-32, or at least one of the combinations 33-48, or at least one of the combinations 49-64:
combination 1: adenine or a derivative thereof-N3-Atto647N; thymine or a derivative thereof-SS-Atto532; cytosine or a derivative thereof-N3-Alexa Fluor532; and guanine or a derivative thereof-SS-CY5;
combination 2: adenine or a derivative thereof-N3-Atto647N; thymine or a derivative thereof-SS-Atto532; cytosine or a derivative thereof-N3-Alexa Fluor532; and guanine or a derivative thereof-SS-Atto647N;
combination 3: adenine or a derivative thereof-N3-Atto647N; thymine or a derivative thereof-SS-Alexa Fluor532; cytosine or a derivative thereof-N3-Atto532; and guanine or a derivative thereof-SS-Atto647N;
combination 4: adenine or a derivative thereof-N3-CY5; thymine or a derivative thereof-SS-Atto532; cytosine or a derivative thereof-N3-Alexa Fluor532; and guanine or a derivative thereof-SS-CY5;
combination 5: adenine or a derivative thereof-N3-CY5; thymine or a derivative thereof-SS-Alexa Fluor532; cytosine or a derivative thereof-N3-Atto532; and guanine or a derivative thereof-SS-CY5;
combination 6: adenine or a derivative thereof-N3-CY5; thymine or a derivative thereof-SS-Atto532; cytosine or a derivative thereof-N3-Atto532; and guanine or a derivative thereof-SS-CY5;
combination 7: adenine or a derivative thereof-N3-CY5; thymine or a derivative thereof-SS-Alexa Fluor532; cytosine or a derivative thereof-N3-Alexa Fluor532; and guanine or a derivative thereof-SS-CY5;
combination 8: adenine or a derivative thereof-N3-Atto647N; thymine or a derivative thereof-SS-Atto532; cytosine or a derivative thereof-N3-Atto532; and guanine or a derivative thereof-SS-CY5;
combination 9: adenine or a derivative thereof-N3-Atto647N; thymine or a derivative thereof-SS-Alexa Fluor532; cytosine or a derivative thereof-N3-Atto532; and guanine or a derivative thereof-SS-CY5;
combination 10: adenine or a derivative thereof-N3-Atto647N; thymine or a derivative thereof-SS-Alexa Fluor532; cytosine or a derivative thereof-N3-Alexa Fluor532; and guanine or a derivative thereof-SS-CY5;
combination 11: adenine or a derivative thereof-N3-CY5; thymine or a derivative thereof-SS-Alexa Fluor532; cytosine or a derivative thereof-N3-Alexa Fluor532; and guanine or a derivative thereof-SS-Atto647N;
combination 12: adenine or a derivative thereof-N3-CY5; thymine or a derivative thereof-SS-Atto532; cytosine or a derivative thereof-N3-Atto532; and guanine or a derivative thereof-SS-Atto647N;
combination 13: adenine or a derivative thereof-N3-CY5; thymine or a derivative thereof-SS-Atto532; cytosine or a derivative thereof-N3-Alexa Fluor532; and guanine or a derivative thereof-SS-Atto647N;
combination 14: adenine or a derivative thereof-N3-CY5; thymine or a derivative thereof-SS-Alexa Fluor532; cytosine or a derivative thereof-N3-Atto532; and guanine or a derivative thereof-SS-Atto647N;
combination 15: adenine or a derivative thereof-N3-Atto647N; thymine or a derivative thereof-SS-Alexa Fluor532; cytosine or a derivative thereof-N3-Alexa Fluor532; and guanine or a derivative thereof-SS-Atto647N;
combination 16: adenine or a derivative thereof-N3-Atto647N; thymine or a derivative thereof-SS-Atto532; cytosine or a derivative thereof-N3-Atto532; and guanine or a derivative thereof-SS-Atto647N;
combination 17: adenine or a derivative thereof-N3-Atto532; thymine or a derivative thereof-SS-Atto647N; cytosine or a derivative thereof-N3-Atto647N; and guanine or a derivative thereof-SS-Atto532;
combination 18: adenine or a derivative thereof-N3-Alexa Fluor532; thymine or a derivative thereof-SS-Atto647N; cytosine or a derivative thereof-N3-Atto647N; and guanine or a derivative thereof-SS-Atto532;
combination 19: adenine or a derivative thereof-N3-Atto532; thymine or a derivative thereof-SS-Atto647N; cytosine or a derivative thereof-N3-Atto647N; and guanine or a derivative thereof-SS-Alexa Fluor532;
combination 20: adenine or a derivative thereof-N3-Alexa Fluor532; thymine or a derivative thereof-SS-Atto647N; cytosine or a derivative thereof-N3-Atto647N; and guanine or a derivative thereof-SS-Alexa Fluor532;
combination 21: adenine or a derivative thereof-N3-Atto532; thymine or a derivative thereof-SS-CY5; cytosine or a derivative thereof-N3-Atto647N; and guanine or a derivative thereof-SS-Atto532;
combination 22: adenine or a derivative thereof-N3-Alexa Fluor532; thymine or a derivative thereof-SS-CY5; cytosine or a derivative thereof-N3-Atto647N; and guanine or a derivative thereof-SS-Atto532;
combination 23: adenine or a derivative thereof-N3-Atto532; thymine or a derivative thereof-SS-CY5; cytosine or a derivative thereof-N3-Atto647N; and guanine or a derivative thereof-SS-Alexa Fluor532;
combination 24: adenine or a derivative thereof-N3-Alexa Fluor532; thymine or a derivative thereof-SS-CY5; cytosine or a derivative thereof-N3-Atto647N; and guanine or a derivative thereof-SS-Alexa Fluor532;
combination 25: adenine or a derivative thereof-N3-Atto532; thymine or a derivative thereof-SS-Atto647N; cytosine or a derivative thereof-N3-CY5; and guanine or a derivative thereof-SS-Atto532;
combination 26: adenine or a derivative thereof-N3-Alexa Fluor532; thymine or a derivative thereof-SS-Atto647N; cytosine or a derivative thereof-N3-CY5; and guanine or a derivative thereof-SS-Atto532;
combination 27: adenine or a derivative thereof-N3-Atto532; thymine or a derivative thereof-SS-Atto647N; cytosine or a derivative thereof-N3-CY5; and guanine or a derivative thereof-SS-Alexa Fluor532;
combination 28: adenine or a derivative thereof-N3-Alexa Fluor532; thymine or a derivative thereof-SS-Atto647N; cytosine or a derivative thereof-N3-CY5; and guanine or a derivative thereof-SS-Alexa Fluor532;
combination 29: adenine or a derivative thereof-N3-Alexa Fluor532; thymine or a derivative thereof-SS-CY5; cytosine or a derivative thereof-N3-CY5; and guanine or a derivative thereof-SS-Atto532;
combination 30: adenine or a derivative thereof-N3-Atto532; thymine or a derivative thereof-SS-CY5; cytosine or a derivative thereof-N3-CY5; and guanine or a derivative thereof-SS-Alexa Fluor532;
combination 31: adenine or a derivative thereof-N3-Atto532; thymine or a derivative thereof-SS-CY5; cytosine or a derivative thereof-N3-CY5; and guanine or a derivative thereof-SS-Atto532; and
combination 32: adenine or a derivative thereof-N3-Alexa Fluor532; thymine or a derivative thereof-SS-CY5; cytosine or a derivative thereof-N3-CY5; and guanine or a derivative thereof-SS-Alexa Fluor532;
combination 33: adenine or a derivative thereof-N3-Atto647N; thymine or a derivative thereof-N3-SS-Atto532; cytosine or a derivative thereof-N3-Alexa Fluor532; and guanine or a derivative thereof-N3-SS-CY5;
combination 34: adenine or a derivative thereof-N3-Atto647N; thymine or a derivative thereof-N3-SS-Atto532; cytosine or a derivative thereof-N3-Alexa Fluor532; and guanine or a derivative thereof-N3-SS-Atto647N;
combination 35: adenine or a derivative thereof-N3-Atto647N; thymine or a derivative thereof-N3-SS-Alexa Fluor532; cytosine or a derivative thereof-N3-Atto532; and guanine or a derivative thereof-N3-SS-Atto647N;
combination 36: adenine or a derivative thereof-N3-CY5; thymine or a derivative thereof-N3-SS-Atto532; cytosine or a derivative thereof-N3-Alexa Fluor532; and guanine or a derivative thereof-N3-SS-CY5;
combination 37: adenine or a derivative thereof-N3-CY5; thymine or a derivative thereof-N3-SS-Alexa Fluor532; cytosine or a derivative thereof-N3-Atto532; and guanine or a derivative thereof-N3-SS-CY5;
combination 38: adenine or a derivative thereof-N3-CY5; thymine or a derivative thereof-N3-SS-Atto532; cytosine or a derivative thereof-N3-Atto532; and guanine or a derivative thereof-N3-SS-CY5;
combination 39: adenine or a derivative thereof-N3-CY5; thymine or a derivative thereof-N3-SS-Alexa Fluor532; cytosine or a derivative thereof-N3-Alexa Fluor532; and guanine or a derivative thereof-N3-SS-CY5;
combination 40: adenine or a derivative thereof-N3-Atto647N; thymine or a derivative thereof-N3-SS-Atto532; cytosine or a derivative thereof-N3-Atto532; and guanine or a derivative thereof-N3-SS-CY5;
combination 41: adenine or a derivative thereof-N3-Atto647N; thymine or a derivative thereof-N3-SS-Alexa Fluor532; cytosine or a derivative thereof-N3-Atto532; and guanine or a derivative thereof-N3-SS-CY5;
combination 42: adenine or a derivative thereof-N3-Atto647N; thymine or a derivative thereof-N3-SS-Alexa Fluor532; cytosine or a derivative thereof-N3-Alexa Fluor532; and guanine or a derivative thereof-N3-SS-CY5;
combination 43: adenine or a derivative thereof-N3-CY5; thymine or a derivative thereof-N3-SS-Alexa Fluor532; cytosine or a derivative thereof-N3-Alexa Fluor532; and guanine or a derivative thereof-N3-SS-Atto647N;
combination 44: adenine or a derivative thereof-N3-CY5; thymine or a derivative thereof-N3-SS-Atto532; cytosine or a derivative thereof-N3-Atto532; and guanine or a derivative thereof-N3-SS-Atto647N;
combination 45: adenine or a derivative thereof-N3-CY5; thymine or a derivative thereof-N3-SS-Atto532; cytosine or a derivative thereof-N3-Alexa Fluor532; and guanine or a derivative thereof-N3-SS-Atto647N;
combination 46: adenine or a derivative thereof-N3-CY5; thymine or a derivative thereof-N3-SS-Alexa Fluor532; cytosine or a derivative thereof-N3-Atto532; and guanine or a derivative thereof-N3-SS-Atto647N;
combination 47: adenine or a derivative thereof-N3-Atto647N; thymine or a derivative thereof-N3-SS-Alexa Fluor532; cytosine or a derivative thereof-N3-Alexa Fluor532; and guanine or a derivative thereof-N3-SS-Atto647N;
combination 48: adenine or a derivative thereof-N3-Atto647N; thymine or a derivative thereof-N3-SS-Atto532; cytosine or a derivative thereof-N3-Atto532; and guanine or a derivative thereof-N3-SS-Atto647N;
combination 49: adenine or a derivative thereof-N3-Atto532; thymine or a derivative thereof-N3-SS-Atto647N; cytosine or a derivative thereof-N3-Atto647N; and guanine or a derivative thereof-N3-SS-Atto532;
combination 50: adenine or a derivative thereof-N3-Alexa Fluor532; thymine or a derivative thereof-N3-SS-Atto647N; cytosine or a derivative thereof-N3-Atto647N; and guanine or a derivative thereof-N3-SS-Atto532;
combination 51: adenine or a derivative thereof-N3-Atto532; thymine or a derivative thereof-N3-SS-Atto647N; cytosine or a derivative thereof-N3-Atto647N; and guanine or a derivative thereof-N3-SS-Alexa Fluor532;
combination 52: adenine or a derivative thereof-N3-Alexa Fluor532; thymine or a derivative thereof-N3-SS-Atto647N; cytosine or a derivative thereof-N3-Atto647N; and guanine or a derivative thereof-N3-SS-Alexa Fluor532;
combination 53: adenine or a derivative thereof-N3-Atto532; thymine or a derivative thereof-N3-SS-CY5; cytosine or a derivative thereof-N3-Atto647N; and guanine or a derivative thereof-N3-SS-Atto532;
combination 54: adenine or a derivative thereof-N3-Alexa Fluor532; thymine or a derivative thereof-N3-SS-CY5; cytosine or a derivative thereof-N3-Atto647N; and guanine or a derivative thereof-N3-SS-Atto532;
combination 55: adenine or a derivative thereof-N3-Atto532; thymine or a derivative thereof-N3-SS-CY5; cytosine or a derivative thereof-N3-Atto647N; and guanine or a derivative thereof-N3-SS-Alexa Fluor532;
combination 56: adenine or a derivative thereof-N3-Alexa Fluor532; thymine or a derivative thereof-N3-SS-CY5; cytosine or a derivative thereof-N3-Atto647N; and guanine or a derivative thereof-N3-SS-Alexa Fluor532;
combination 57: adenine or a derivative thereof-N3-Atto532; thymine or a derivative thereof-N3-SS-Atto647N; cytosine or a derivative thereof-N3-CY5; and guanine or a derivative thereof-N3-SS-Atto532;
combination 58: adenine or a derivative thereof-N3-Alexa Fluor532; thymine or a derivative thereof-N3-SS-Atto647N; cytosine or a derivative thereof-N3-CY5; and guanine or a derivative thereof-N3-SS-Atto532;
combination 59: adenine or a derivative thereof-N3-Atto532; thymine or a derivative thereof-N3-SS-Atto647N; cytosine or a derivative thereof-N3-CY5; and guanine or a derivative thereof-N3-SS-Alexa Fluor532;
combination 60: adenine or a derivative thereof-N3-Alexa Fluor532; thymine or a derivative thereof-N3-SS-Atto647N; cytosine or a derivative thereof-N3-CY5; and guanine or a derivative thereof-N3-SS-Alexa Fluor532;
combination 61: adenine or a derivative thereof-N3-Alexa Fluor532; thymine or a derivative thereof-N3-SS-CY5; cytosine or a derivative thereof-N3-CY5; and guanine or a derivative thereof-N3-SS-Atto532;
combination 62: adenine or a derivative thereof-N3-Atto532; thymine or a derivative thereof-N3-SS-CY5; cytosine or a derivative thereof-N3-CY5; and guanine or a derivative thereof-N3-SS-Alexa Fluor532;
combination 63: adenine or a derivative thereof-N3-Atto532; thymine or a derivative thereof-N3-SS-CY5; cytosine or a derivative thereof-N3-CY5; and guanine or a derivative thereof-N3-SS-Atto532; and
combination 64: adenine or a derivative thereof-N3-Alexa Fluor532; and thymine or a derivative thereof-N3-SS-CY5.

Based on the above embodiments, as one possible implementation of the kit of the present application, cleavable groups in the above combinations may be replaced;
in combinations 1-32 of the above cases, —N3- linked to adenine or a derivative thereof is replaced with —SS—, —N3- linked to cytosine or a derivative thereof-N3 is replaced with —SS—, —SS— linked to thymine or a derivative thereof is replaced with —N3-, and —SS— linked to guanine or a derivative thereof is replaced with —N3-.

As one possible implementation of the kit of the present application, in combinations 1-32 of the above cases, —N3- linked to adenine or a derivative thereof is replaced with —SS—, and —SS— linked to guanine or a derivative thereof is replaced with —N3-.

As one possible implementation of the kit of the present application, in combinations 1-32 of the above cases, —SS— linked to thymine or a derivative thereof is replaced with —N3-, and —N3- linked to cytosine or a derivative thereof is replaced with —SS—.

As one possible implementation of the kit of the present application, in combinations 33-64 of the above cases, a single cleavable group-N3- linked to adenine or a derivative thereof is replaced with a double cleavable group-N3-SS—, a single cleavable group-N3- linked to cytosine or a derivative thereof is replaced with —N3-SS—, a double cleavable group-N3-SS— linked to thymine or a derivative thereof is replaced with a single cleavable group-N3-, and a double cleavable group-N3-SS— linked to guanine or a derivative thereof is replaced with a single cleavable group-N3-; or
as one possible implementation of the kit of the present application, in combinations 33-64 of the above cases, a single cleavable group-N3- linked to adenine or a derivative thereof is replaced with a double cleavable group-N3-SS—, and a double cleavable group-N3-SS— linked to guanine or a derivative thereof is replaced with a single cleavable group-N3-; or
as one possible implementation of the kit of the present application, in combinations 33-64 of the above cases, a double cleavable group-N3-SS— linked to thymine or a derivative thereof is replaced with a single cleavable group-N3-, and a single cleavable group-N3- linked to cytosine or a derivative thereof is replaced with a double cleavable group-N3-SS—.

As one possible implementation of the kit of the present application, in combinations 1-64 of the above cases or after replacing the cleavable groups in combinations 1-64, CY5 and/or Atto647N is replaced with GM656 or Alexa Fluor647; and/or Atto532 and/or Alexa Fluor532 is replaced with Atto Rho6G or CY3.

As one possible implementation of the kit of the present application, in combinations 1-64 of the above cases or after replacing the cleavable groups in combinations 1-64, CY5 and/or Atto647N is replaced with one type of blue-emitting fluorescent dyes.

As one possible implementation of the kit of the present application, the reversible terminators are combination 1.

As one possible implementation of the kit of the present application, the working concentrations of the reversible terminators are: adenine nucleotide or the derivative thereof-N3-Atto647N at 10 nmol/L-700 nmol/L; thymine nucleotide or the derivative thereof-N3-SS-Atto532 at 5 nmol/L-60 nmol/L; cytosine nucleotide or the derivative thereof-N3-Alexa Fluor532 at 10 nmol/L-700 nmol/L; and guanine nucleotide or the derivative thereof-N3-SS-CY5 at 0.05 nmol/L-5 nmol/L.

As one possible implementation of the kit of the present application, in the organic phosphine cleavage reagent, the organic phosphine is selected from any one or more of tris(3-hydroxypropyl)phosphine, tris(2-carboxyethyl)phosphine, tris(hydroxymethyl)phosphine, triphenylphosphine, or tris(2-hydroxyethyl)phosphine; and
as one possible implementation of the kit of the present application, in the organic phosphine cleavage reagent, the organic phosphine is tris(3-hydroxypropyl)phosphine.

As one possible implementation of the kit of the present application, the working concentration of the tris(3-hydroxypropyl)phosphine is 10 mmol/L-100 mmol/L.

As one possible implementation of the kit of the present application, the reducing agent is selected from at least one of DTT, glutathione, and β-mercaptoethanol.

As one possible implementation of the kit of the present application, in the reducing agent cleavage reagent, the reducing agent is glutathione.

As one possible implementation of the kit of the present application, the working concentration of the glutathione is 10 mmol/L-100 mmol/L.

Through the optimization of the concentration ratios of the reaction substances and the reaction parameters, the present disclosure develops a two-color sequencing method suitable for a single-molecule sequencing platform. The two-color sequencing method is used for single-molecule sequencing, and is beneficial for reducing error rate, and improving sequencing quality.

### BRIEF DESCRIPTION OF THE DRAWINGS

FIG. 1 shows the adsorption of 500 nmol/L dA-N3-Atto647N (left) and the adsorption of 10 nmol/L dA-N3-Atto647N (right) in two-color dual-cleavage sequencing;

FIG. 2 shows the disulfide bond cleavage efficiency in dG-SS-Atto647N under treatment with GSH at different concentrations;

FIG. 3 shows the cleavage efficiency of azide groups on the bases of dA-N3-Atto647N and dC-N3-Alexa Fluor532 by THPP in two-color dual-cleavage sequencing;

FIG. 4 shows a process of two-color dual-cleavage sequencing; and

FIG. 5 shows a read length distribution of 50-cycle two-color dual-cleavage sequencing.

### DETAILED DESCRIPTION OF THE INVENTION

In order to make the technical problems, technical schemes, and beneficial effects to be solved by the present application more apparent, the present application will be further described in detail below with reference to the embodiments. It should be understood that the specific embodiments described herein are intended to illustrate the present application only, rather than limit the present application.

In the present application, the terms used in the embodiments of the present application are for the purpose of describing particular embodiments only and are not intended to be limiting to the present application. The term “and/or” describes the associative relationship between associated objects, and refers to three possible relationships. For example, A and/or B may denote that: A is present alone, A and B are present simultaneously, and B is present alone. A and B may be singular or plural. The character “/” in “and/or” denotes that the former and latter associated objects are in an “and” relationship.

The term “at least one” means one or more, and “a plurality of” means two or more. The “at least one” or similar expressions thereof refer to any combination of these items, including any combination of the singular or plural items. For example, “at least one of a, b, or c” or “at least one of a, b, and c” may each refer to: a, b, c, a-b (i.e., a and b), a-c, b-c, or a-b-c, where a, b, and c may be a single item or a plurality of items, respectively.

As used in the embodiments and the appended claims of the present application, the singular forms “a”, “an”, and “the” are intended to include the plural forms as well, unless the context clearly indicates otherwise.

The terms “first” and “second” are used for descriptive purposes only, are used for distinguishing purposes such as substances or objects from one another, and should not be understood as indicating or implying relative importance or implicitly indicating the number of technical features indicated. For example, without departing from the scope of the embodiments of the present application, the first reversible terminator may also be referred to as the second reversible terminator, and the second reversible terminator is referred to as the first reversible terminator. Therefore, features defined by “first” and “second” may explicitly or implicitly include one or more of the features.

It should be understood that in the embodiments of the present application, the serial numbers of the processes described above do not imply an execution sequence, some or all of the steps may be executed in parallel or executed sequentially, and the execution sequence of each process should be determined by its function and inherent logic and should not constitute any limitation to the implementation process of the embodiments of the present application.

In the embodiments of the present application, the term “sequencing” may also be referred to as “nucleic acid sequencing” or “gene sequencing”. That is, the three are used interchangeably to refer to the determination of the type and order of bases in a nucleic acid sequence, including the procedures of binding nucleotides (including nucleotide analogs) to a template and acquiring the corresponding signals emitted by the nucleotides (including analogs). The “sequencing” includes sequencing by synthesis (SBS) and/or sequencing by ligation (SBL); includes DNA sequencing and/or RNA sequencing; includes long fragment sequencing and/or short fragment sequencing (the long fragment and short fragment are defined relatively; for example, nucleic acid molecules longer than 1 Kb, 2 Kb, 5 Kb or 10 Kb may be referred to as long fragments, and nucleic acid molecules shorter than 1 Kb or 800 bp may be referred to as short fragments); and includes double-end sequencing, single-end sequencing, paired-end sequencing, and/or the like, where the double-end sequencing or paired-end sequencing may refer to the reading of any two segments or portions of the same nucleic acid molecule that are not completely overlapping.

The sequencing generally involves multiple cycles of process to achieve the determination of the types and sequence of multiple bases or nucleotides on the nucleic acid template. The embodiments of the present application refer to each cycle of the “process to achieve the determination of the types and sequence of multiple bases or nucleotides on the nucleic acid template” as one “cycle of sequencing”. The “cycle of sequencing”, also known as “sequencing cycle”, may be defined as the completion of one base extension of the four types of nucleotides/bases; in other words, one “cycle of sequencing” may be defined as the determination of the base or nucleotide type at any given position on the template. For sequencing platforms that achieve sequencing on the basis of polymerization or ligation reactions, one cycle of sequencing includes the process of binding four types of nucleotides (including nucleotide analogs) to the nucleic acid template at a time according to the base complementary rule and acquiring the corresponding signals emitted. For platforms that achieve sequencing based on the polymerase chain reaction, a reaction system includes a nucleotide as a reaction substrate, a polymerase, and a nucleic acid template. A sequence fragment (a sequencing primer) binds to the nucleic acid template, and based on the base pairing rules and the principle of polymerase chain reaction, the nucleotide added as the reaction substrate is ligated to the sequencing primer under the catalysis of the polymerase to achieve the binding of the nucleotide to a specific position on the nucleic acid template. Generally, one cycle of sequencing may include one or more base extensions (repeats). For example, four types of nucleotides are sequentially added to the reaction system to each perform base extensions and corresponding acquisition of reaction signals, and one cycle of sequencing includes four base extensions; for another example, four types of nucleotides are added to the reaction system in any combinations (such as in pairs or in one-three combinations), the two combinations each perform base extensions and corresponding acquisition of reaction signals, and one cycle of sequencing includes two base extensions; for yet another example, four types of nucleotides are added simultaneously to the reaction system for base extension and acquisition of reaction signals, and one cycle of sequencing includes one base extension.

The term “nucleotide” refers to a molecule including a sugar, at least one phosphate group, and a nucleobase. The nucleotides include deoxyribonucleotides, modified deoxyribonucleotides, ribonucleotides, modified ribonucleotides, peptide nucleotides, modified peptide nucleotides, modified sugar-phosphate backbone nucleotides, and mixtures thereof. Examples of the nucleotide include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” is also intended to encompass any nucleotide analogs that are those including a modified nucleobase, sugar, and/or phosphate moiety as compared to naturally occurring nucleotides. Exemplary modified nucleobases include inosine, xathanine, hypoxanthine, isocytosine, isoguanine, 2-aminopurine, 5-methylcytosine, 5-hydoxymethylcytosine, 2-aminoadenine, 6-methyladenine, 6-methylguanine, 2-propylguanine, 2-propyladenine, 2-thiouracil, 2-thiothymine, 2-thiocytosine, 15-halouracil, 15-halocytosine, 5-propynyluracil, 5-propynylcytosine, 6-azouracil, 6-azocytosine, 6-azothymine, 5-uracil, 4-thiouracil, 8-haloadenine or guanine, 8-aminoadenine or guanine, 8-thioladenine or guanine, 8-sulfanyladenine or guanine, 8-hydoxyadenine or guanine, 5-halouracil or cytosine, 7-methylguanine, 7-methyladenine, 8-azaguanine, 8-azaadenine, 7-deazaguanine, 7-deazaadenine, 3-deazaguanine, 3-deazaadenine, etc. As is known in the art, certain nucleotide analogs cannot be incorporated into polynucleotides, for example, nucleotide analogs such as adenosine 5′-phosphosulfate. The nucleotide may include any suitable number of phosphates, for example, three, four, five, six, or more than six phosphates.

The term “cluster” refers to a population of nucleic acid molecules linked to the surface of the solid substrate to form a feature or site, where such nucleic acid molecules in the population of nucleic acid molecules typically are identical in sequence and can form a single clonal cluster by amplification or cloning. A non-monoclonal cluster mainly includes an amplicon from a first nucleic acid, and may also have a lower level of contaminating amplicons from a second nucleic acid. For example, when using a cluster array in a detection application, an acceptable contamination level is one that does not affect the signal-to-noise ratio or resolution of the detection technique in an unacceptable manner. Thus, apparent clonality is generally associated with the particular use or application of the array prepared by the methods described herein.

The term “primer”, also known as “probe”, refers to an oligonucleotide or a nucleic acid molecule that can hybridize to a target sequence of interest. In the embodiments, the primer serves as a substrate onto which nucleotides may be polymerized by a polymerase. For example, the primer may be used as a starting point for DNA or RNA synthesis. For example, a sequencing primer can hybridize to a synthesized nucleic acid template strand so as to trigger the synthesis of a new strand complementary to the synthesized nucleic acid template strand. The primer may include any combination of nucleotides or analogs thereof. In some examples, the primer is a single-stranded oligonucleotide or a polynucleotide.

The term “FOV” refers to the field of view. The application of the solid substrate includes a process of performing multiple cycles of optical imaging (photographing) on a fixed region of the solid substrate by using an optical camera, and the region photographed by the optical camera each time is referred to as a FOV (field of view).

In the embodiments of the present application, —N3- is an azide group containing azidomethylene (—CH₂(N₃)—); and —SS— is a chemical structure or a chemical group containing disulfide bonds.

The use of the terms “comprise”, “include”, “have”, or “contain”, including grammatical synonyms thereof, should generally be understood as open-ended and non-limiting, for example, without excluding other unrecited elements or steps, unless otherwise specifically stated or unless otherwise understood from the context.

The use of any and all examples or exemplary language herein, such as “for example” or “including”, is merely intended to better illustrate the present disclosure and does not pose a limitation on the scope of the present disclosure unless claimed. The language in this specification should not be construed as indicating any unclaimed elements as essential for the practice of the present disclosure.

In addition, the numerical ranges and parameters defining the present disclosure are approximations, and the related numerical values in the specific embodiments have been presented as precisely as possible. Any numerical value, however, inherently contains a standard deviation necessarily resulting from certain methods of testing. Accordingly, unless otherwise explicitly indicated, it should be understood that all ranges, quantities, numerical values, and percentages used herein are modified by the word “about”. Herein, “about” generally means that the actual numerical value is within a particular numerical value or range ±10%, 5%, 1%, or 0.5%.

Taking the sequencing by synthesis as an example, the two-color sequencing method is described below.

The two-color sequencing method provided by the embodiments of the present application includes the following steps:

In step 1, four types of reversible terminators: a first reversible terminator, a second reversible terminator, a third reversible terminator, and a fourth reversible terminator are provided, where the four types of reversible terminators each independently include: a nucleotide or a derivative thereof, a fluorophore, and a first cleavable group and/or a second cleavable group linking the nucleotide or the derivative thereof and the fluorophore.

The nucleotide or the derivative thereof is selected from adenine or a derivative thereof, thymine or a derivative thereof, cytosine or a derivative thereof, or guanine or a derivative thereof, and the fluorophore is selected from a first fluorophore and a second fluorophore that are distinguishable in color. That is, the sequencing method provided by the embodiments of the present application is a method for achieving base calling by using fluorescence signals of two different colors.

In step 2, the reversible terminators are bound to a nucleic acid template-primer complex by a polymerase chain reaction, and the primer is extended to obtain an extension product;
the binding of the reversible terminators to the nucleic acid template-primer complex and the extension occur on a solid carrier by a polymerase chain reaction, the solid carrier includes a plurality of sites, and each of the sites has a nucleic acid template-primer complex immobilized thereon.

In this step, the solid carrier is understood to be a carrier or support on which a nucleic acid molecule or nucleic acid template is immobilized. In some application examples, the solid carrier may also be referred to as a solid substrate, a sequencing chip, or a sequencing biochip. In one embodiment, the solid carrier is a substrate with an array of micropores, and each micropore is located at a position corresponding to a site. Illustratively, the solid carrier is a nanowell pattern chip, and the nanowell pattern chip includes nanowells arranged in an array for immobilizing the nucleic acid template-primer complex, where the nanowell may also be referred to as a nanopore, a micropore, or a pore. In the embodiments of the present application, the indicated nanowell and nanopore may be interchangeable, and refer to a nanoscale pore structure formed on the surface of the solid carrier. The shape of the nanowell or nanopore is not strictly limited, and the nanowell or nanopore may have a shape of a circle, an ellipse, a triangle, a quadrangle or other polygons, or even has no specific shape. In some embodiments, the nanowell or nanopore is configured to be a shape with a symmetrical cross-section. Illustratively, the cross-section of the nanowell or nanopore is a circle or regular polygon.

In one embodiment, the solid carrier is a planar substrate, and the surface of the planar substrate includes several surface-processed positions, each position corresponding to a site for immobilizing the nucleic acid template-primer complex. Surface processing refers to forming an active functional group, which can be linked to the nucleic acid template-primer complex, on the surface. An example of this type of solid carrier is a random chip.

The solid carrier has a plurality of sites, and there is a certain distance between two adjacent sites. The distance is large enough such that optical signals generated by fluorophores at the adjacent sites can be distinguished.

In this embodiment, a nucleic acid template is immobilized on the surface of the solid carrier, and the nucleic acid template is linked to the surface of the solid carrier. In one embodiment, a probe is linked to the surface of the solid carrier, the probe is linked to a molecule/group on the surface of the solid carrier via a covalent bond, and the nucleic acid template is linked to the surface of the solid carrier via the probe. The probe is an oligonucleotide or nucleic acid molecule fragment that can hybridize to a target nucleic acid molecule of interest. In one embodiment, at least a portion of the probe is configured to hybridize to at least a portion of the 3′ end of the nucleic acid template. In another embodiment, one end of the nucleic acid template is linked to a molecule/group on the surface of the solid carrier via a covalent bond, thereby linking to the surface of the solid carrier. In the embodiments of the present application, the probe and the nucleic acid template jointly form the nucleic acid template-primer complex.

In this embodiment, the solid carrier has a plurality of sites, and correspondingly, the nucleic acid template is immobilized on the site of the solid carrier. According to the embodiments of the present application, ideally, one type of nucleic acid template is immobilized at each site. In some inevitable cases, there may be cases where very few sites have no nucleic acid template immobilized or where two types of nucleic acid templates are immobilized at one site. When the same site contains two different nucleic acid templates, the site produces two detectable signals in one cycle of sequencing. To improve the accuracy of sequencing, the sequencing signals at these sites are not counted, and the corresponding sequencing data are discarded.

In some embodiments, one nucleic acid molecule is immobilized at each site on the surface of the solid carrier, and in this case, the two-color sequencing provided by the embodiments of the present application is single-molecule sequencing. In some other embodiments, each site on the surface of the solid carrier includes a plurality of amplicons of one nucleic acid molecule, that is, each site includes one nucleic acid molecule cluster, and in this case, the two-color sequencing provided by the embodiments of the present application is sequencing provided for the nucleic acid molecule cluster.

In some embodiments, the solid carrier includes a channel structure that can communicate one end of the solid carrier to the other end, and in this case, the solution and/or reagent for the polymerase chain reaction in step 2 and other solutions subsequently contacted with the solid carrier can be contacted by flowing through the surface of the channel structure. In one embodiment, the channel structure on the surface of the solid carrier may be formed on the surface of the solid carrier by, for example, etching. Materials are removed from a portion of the surface of the solid carrier by, for example, etching, such that the portion of the surface forms a groove structure. The groove structure leads from one end of the solid carrier to the other end of the solid carrier. The channel structure on the surface of the solid carrier can also be formed by covering a mask material layer on the surface of a solid substrate with a certain thickness, and the pattern of the mask material layer corresponds to the expected channel structure arranged on the surface of the solid carrier. For example, the mask material layer masks a region where the corresponding groove is located, hollow processing is performed on the other regions except the groove region, and then the material is deposited on the hollowed region of the mask material layer by using, for example, a chemical deposition method, so as to form a material layer with the channel structure on the surface of the solid carrier, and the solid carrier and the material layer formed on the surface of the solid carrier jointly constitute the solid carrier. It should be understood that the surface of the solid carrier may have one channel structure, or two or more, such as 4, 8, or 16, channel structures. Certainly, the number of channel structures is not limited thereto. In some embodiments, the channel structure on the surface of the solid carrier is also referred to as Lane.

Illustratively, the channel structure has a width of 5 mmol/L-15 mmol/L and a height of 50 μm-200 μm. It should be understood that the width and height of the solid carrier may be arbitrarily combined within the ranges described above without strict limitations. Illustratively, when the width of the solid carrier is 5 mmol/L-10 mmol/L, the height of the solid carrier may be in the range of 50 μm-100 μm, 80 μm-120 μm, 100 μm-150 μm, 120 μm-180 μm, 180 μm-200 μm, or the like; certainly, when the width of the solid carrier is 10 mmol/L-15 mmol/L, the height of the solid carrier may also be in the range of 50 μm-100 μm, 80 μm-120 μm, 100 μm-150 μm, 120 μm-180 μm, 180 μm-200 μm, or the like.

In step 2, four types of different nucleotides or analogs thereof carrying fluorophores and different cleavable groups are bound to the nucleic acid template-primer complex by a polymerase chain reaction to achieve single-base extension and obtain an extension product.

In this step, four types of nucleotides or analogs thereof (hereinafter referred to as “reversible terminators”) carrying fluorophores and cleavable groups are added to the surface of the solid carrier under conditions suitable for a polymerase chain reaction, such that the reversible terminators can pair with the nucleic acid template by the base complementary pairing rules and bind to the nucleic acid template-primer complex. In the embodiments of the present application, the four types of reversible terminators are uniformly dispersed through a medium and are brought into contact with the nucleic acid template-primer complex. The medium used for dispersing the four types of nucleotides or the analogs thereof may be a liquid medium, such as a reagent used for dissolving the four types of reversible terminators, the polymerase, and the like. In one embodiment, the four types of reversible terminators and the polymerase are dissolved in a reagent, and under conditions suitable for a polymerization reaction, the reversible terminators are contacted with the nucleic acid template-primer complex to perform a polymerase chain reaction, thereby incorporating one reversible terminator into the primer strand.

For ease of distinguishing the four types of reversible terminators, in the embodiments of the present application, the four types of reversible terminators are respectively named as: the first reversible terminator, the second reversible terminator, the third reversible terminator, and the fourth reversible terminator. The four types of reversible terminators each independently include: a nucleotide or a derivative thereof, a fluorophore, and a first cleavable group and/or a second cleavable group linking the nucleotide or the derivative thereof and the fluorophore.

To distinguish the two types of cleavable groups, in the embodiments of the present application, a cleavable group with stronger cleavability is defined as the second cleavable group, and compared with the second cleavable group, the first cleavable group has weaker cleavability. It should be understood that in the embodiments of the present application, cleavability refers to the activity by which a group can be separated from a nucleotide or an analog thereof under the same chemical, biological, or physical condition. The manner of separation may be, but is not limited to, chemical cleavage, enzymatic cleavage, photocleavage, or the like, and the strength of cleavability may be reflected by whether cleavage can occur, the time required for cleavage, and the like. In the embodiments of the present application, the difference in cleavability between the first cleavable group and the second cleavable group satisfies that: under the same chemical, biological, or physical condition, the second cleavable group is cleaved from the structure of the nucleotide or the derivative thereof, while the first cleavable group still remains in the structure of the nucleotide or the derivative thereof.

In some embodiments, the first cleavable group is selected from a group including at least one of —N3-, —CH₂F, —C(O)NHBu, —CHF₂, —C(O)OH, —C₆, and/or —ArC₆. In some embodiments, the first cleavable group is selected from at least one of —N3-, —CH₂F, —C(O)NHBu, —CHF₂, —C(O)OH, —C₆, and/or —ArC₆. In some embodiments, the second cleavable group is selected from a group including a disulfide bond, such as a disulfide bond.

In the embodiments of the present application, the fluorophore refers to a group capable of absorbing photons with a certain energy (wavelength) and, upon optical excitation, generating a detectable optical signal, where the optical signal includes a fluorescence signal. The four types of reversible terminators provided by the embodiments of the present application all carry fluorophores, and the fluorophores carried by the four types of reversible terminators have two distinguishable colors, that is, the fluorophores on the four types of reversible terminators are selected from a first fluorophore and a second fluorophore that are distinguishable in color.

In some embodiments, the fluorescence color of the first fluorophore is red, green, or blue, and the fluorescence color of the second fluorophore is red, green, or blue. Certainly, under the fluorescence signal acquisition conditions provided in the embodiments of the present application, the fluorescence colors of the first fluorophore and the second fluorophore are different. In one embodiment, the combination of the first fluorescence signal and the second fluorescence signal is a combination formed by red and green fluorescence signals; or the combination of the first fluorescence signal and the second fluorescence signal is a combination formed by green and blue fluorescence signals.

In some embodiments, the first fluorophore and the second fluorophore are each independently selected from one of or a combination of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin.

The red fluorophore includes Atto647N, CY5, GM656, or Alexa Fluor647;
the fluorophore having a fluorescence color of blue includes: Atto532, Alexa Fluor532, Atto Rho6G, or CY3;
the green fluorophore includes: Atto647N, CY5, CY2, IF700, Alexa Fluor®430, and Alexa Fluor®488; and
the blue fluorophore includes: Atto390, Alexa Fluor405, or coumarin.

In some embodiments, the four types of reversible terminators are combined with different cleavable groups and/or different fluorophores. After the second cleavable group is cleaved in the first cleavage reaction, at least a portion of the four types of reversible terminators (hereinafter referred to as the first portion) can be distinguished from the other portion of the four types of reversible terminators (hereinafter referred to as the second portion). After the first cleavable group is cleaved in the second cleavage reaction, the first portion of the four types of reversible terminators can be distinguished from one another, and the second portion can also be distinguished from one another, thereby ultimately achieving stepwise and individual discrimination of the signals of the four types of reversible terminators using two-color fluorescence. Illustratively, any two types of the four types of reversible terminators have the same cleavable group, and the fluorophores of the reversible terminators having the same cleavable group have different fluorescence colors.

As one implementation of the above embodiments, the structures of the four types of reversible terminators are as follows:
the structure of the first reversible terminator is: a first nucleotide or a derivative thereof-the first cleavable group-the first fluorophore;
the structure of the second reversible terminator is: a second nucleotide or a derivative thereof-the second cleavable group-the first fluorophore;
the structure of the third reversible terminator is: a third nucleotide or a derivative thereof-the second cleavable group-the second fluorophore; and
the structure of the fourth reversible terminator is: a fourth nucleotide or a derivative thereof-the first cleavable group-the second fluorophore,
where the first nucleotide or the derivative thereof, the second nucleotide or the derivative thereof, the third nucleotide or the derivative thereof, and the fourth nucleotide or the derivative thereof are independently selected from one of adenine or a derivative thereof, thymine or a derivative thereof, cytosine guanine or a derivative thereof, and guanine or a derivative thereof, and the first nucleotide or the derivative thereof, the second nucleotide or the derivative thereof, the third nucleotide or the derivative thereof, and the fourth nucleotide or the derivative thereof are different from one another.

In the embodiments of the present application, the combination of the first cleavable group and the second cleavable group satisfies that: under the same cleavage condition, when the second cleavable group is cleaved, the first cleavable group is not affected and still remains in the reversible terminator structure. In some embodiments, the first cleavable group in the above four types of reversible terminators is —N3-, and the second cleavable group is-SS—. Certainly, the combination of the first cleavable group and the second cleavable group is not limited thereto.

Illustratively, the four types of reversible terminators include at least one of the following combination 1 to combination 16; or the four types of reversible terminators include at least one of the following combination 17 to combination 32:
combination 1: adenine or a derivative thereof-N3-Atto647N; thymine or a derivative thereof-SS-Atto532; cytosine or a derivative thereof-N3-Alexa Fluor532; and guanine or a derivative thereof-SS-CY5;
combination 2: adenine or a derivative thereof-N3-Atto647N; thymine or a derivative thereof-SS-Atto532; cytosine or a derivative thereof-N3-Alexa Fluor532; and guanine or a derivative thereof-SS-Atto647N;
combination 3: adenine or a derivative thereof-N3-Atto647N; thymine or a derivative thereof-SS-Alexa Fluor532; cytosine or a derivative thereof-N3-Atto532; and guanine or a derivative thereof-SS-Atto647N;
combination 4: adenine or a derivative thereof-N3-CY5; thymine or a derivative thereof-SS-Atto532; cytosine or a derivative thereof-N3-Alexa Fluor532; and guanine or a derivative thereof-SS-CY5;
combination 5: adenine or a derivative thereof-N3-CY5; thymine or a derivative thereof-SS-Alexa Fluor532; cytosine or a derivative thereof-N3-Atto532; and guanine or a derivative thereof-SS-CY5;
combination 6: adenine or a derivative thereof-N3-CY5; thymine or a derivative thereof-SS-Atto532; cytosine or a derivative thereof-N3-Atto532; and guanine or a derivative thereof-SS-CY5;
combination 7: adenine or a derivative thereof-N3-CY5; thymine or a derivative thereof-SS-Alexa Fluor532; cytosine or a derivative thereof-N3-Alexa Fluor532; and guanine or a derivative thereof-SS-CY5;
combination 8: adenine or a derivative thereof-N3-Atto647N; thymine or a derivative thereof-SS-Atto532; cytosine or a derivative thereof-N3-Atto532; and guanine or a derivative thereof-SS-CY5;
combination 9: adenine or a derivative thereof-N3-Atto647N; thymine or a derivative thereof-SS-Alexa Fluor532; cytosine or a derivative thereof-N3-Atto532; and guanine or a derivative thereof-SS-CY5;
combination 10: adenine or a derivative thereof-N3-Atto647N; thymine or a derivative thereof-SS-Alexa Fluor532; cytosine or a derivative thereof-N3-Alexa Fluor532; and guanine or a derivative thereof-SS-CY5;
combination 11: adenine or a derivative thereof-N3-CY5; thymine or a derivative thereof-SS-Alexa Fluor532; cytosine or a derivative thereof-N3-Alexa Fluor532; and guanine or a derivative thereof-SS-Atto647N;
combination 12: adenine or a derivative thereof-N3-CY5; thymine or a derivative thereof-SS-Atto532; cytosine or a derivative thereof-N3-Atto532; and guanine or a derivative thereof-SS-Atto647N;
combination 13: adenine or a derivative thereof-N3-CY5; thymine or a derivative thereof-SS-Atto532; cytosine or a derivative thereof-N3-Alexa Fluor532; and guanine or a derivative thereof-SS-Atto647N;
combination 14: adenine or a derivative thereof-N3-CY5; thymine or a derivative thereof-SS-Alexa Fluor532; cytosine or a derivative thereof-N3-Atto532; and guanine or a derivative thereof-SS-Atto647N;
combination 15: adenine or a derivative thereof-N3-Atto647N; thymine or a derivative thereof-SS-Alexa Fluor532; cytosine or a derivative thereof-N3-Alexa Fluor532; and guanine or a derivative thereof-SS-Atto647N;
combination 16: adenine or a derivative thereof-N3-Atto647N; thymine or a derivative thereof-SS-Atto532; cytosine or a derivative thereof-N3-Atto532; and guanine or a derivative thereof-SS-Atto647N;
combination 17: adenine or a derivative thereof-N3-Atto532; thymine or a derivative thereof-SS-Atto647N; cytosine or a derivative thereof-N3-Atto647N; and guanine or a derivative thereof-SS-Atto532;
combination 18: adenine or a derivative thereof-N3-Alexa Fluor532; thymine or a derivative thereof-SS-Atto647N; cytosine or a derivative thereof-N3-Atto647N; and guanine or a derivative thereof-SS-Atto532;
combination 19: adenine or a derivative thereof-N3-Atto532; thymine or a derivative thereof-SS-Atto647N; cytosine or a derivative thereof-N3-Atto647N; and guanine or a derivative thereof-SS-Alexa Fluor532;
combination 20: adenine or a derivative thereof-N3-Alexa Fluor532; thymine or a derivative thereof-SS-Atto647N; cytosine or a derivative thereof-N3-Atto647N; and guanine or a derivative thereof-SS-Alexa Fluor532;
combination 21: adenine or a derivative thereof-N3-Atto532; thymine or a derivative thereof-SS-CY5; cytosine or a derivative thereof-N3-Atto647N; and guanine or a derivative thereof-SS-Atto532;
combination 22: adenine or a derivative thereof-N3-Alexa Fluor532; thymine or a derivative thereof-SS-CY5; cytosine or a derivative thereof-N3-Atto647N; and guanine or a derivative thereof-SS-Atto532;
combination 23: adenine or a derivative thereof-N3-Atto532; thymine or a derivative thereof-SS-CY5; cytosine or a derivative thereof-N3-Atto647N; and guanine or a derivative thereof-SS-Alexa Fluor532;
combination 24: adenine or a derivative thereof-N3-Alexa Fluor532; thymine or a derivative thereof-SS-CY5; cytosine or a derivative thereof-N3-Atto647N; and guanine or a derivative thereof-SS-Alexa Fluor532;
combination 25: adenine or a derivative thereof-N3-Atto532; thymine or a derivative thereof-SS-Atto647N; cytosine or a derivative thereof-N3-CY5; and guanine or a derivative thereof-SS-Atto532;
combination 26: adenine or a derivative thereof-N3-Alexa Fluor532; thymine or a derivative thereof-SS-Atto647N; cytosine or a derivative thereof-N3-CY5; and guanine or a derivative thereof-SS-Atto532;
combination 27: adenine or a derivative thereof-N3-Atto532; thymine or a derivative thereof-SS-Atto647N; cytosine or a derivative thereof-N3-CY5; and guanine or a derivative thereof-SS-Alexa Fluor532;
combination 28: adenine or a derivative thereof-N3-Alexa Fluor532; thymine or a derivative thereof-SS-Atto647N; cytosine or a derivative thereof-N3-CY5; and guanine or a derivative thereof-SS-Alexa Fluor532;
combination 29: adenine or a derivative thereof-N3-Alexa Fluor532; thymine or a derivative thereof-SS-CY5; cytosine or a derivative thereof-N3-CY5; and guanine or a derivative thereof-SS-Atto532;
combination 30: adenine or a derivative thereof-N3-Atto532; thymine or a derivative thereof-SS-CY5; cytosine or a derivative thereof-N3-CY5; and guanine or a derivative thereof-SS-Alexa Fluor532;
combination 31: adenine or a derivative thereof-N3-Atto532; thymine or a derivative thereof-SS-CY5; cytosine or a derivative thereof-N3-CY5; and guanine or a derivative thereof-SS-Atto532; and
combination 32: adenine or a derivative thereof-N3-Alexa Fluor532; thymine or a derivative thereof-SS-CY5; cytosine or a derivative thereof-N3-CY5; and guanine or a derivative thereof-SS-Alexa Fluor532.

As another implementation, in the above combinations 1-32, —N3- linked to the purine or the derivative thereof or the pyrimidine or the derivative thereof is replaced with —SS—, and —SS-linked to the purine or the derivative thereof or the pyrimidine or the derivative thereof is replaced with —N3-. In this case, due to the weak reducibility of —SS—, the first fluorophore and the second fluorophore in two types of the reversible terminators having the cleavable group-SS-in the structure may be preferentially cleaved along with the cleavage of —SS—. Identification of the four types of reversible terminators can be achieved by comparing changes in fluorescence signal before and after cleavage of —SS—.

Similarly, in some embodiments, in combinations 1-32, —N3- linked to adenine or the derivative thereof is replaced with —SS—, and —SS— linked to guanine or the derivative thereof is replaced with —N3-. In some embodiments, in combinations 1-32, —SS— linked to thymine or the derivative thereof is replaced with —N3-, and —N3- linked to cytosine or the derivative thereof is replaced with —SS—.

The above combinations 1-32 provide a plurality of reversible terminator combinations that include red fluorophores and green fluorophores. Certainly, the combinations of red fluorophores and green fluorophores are not limited to the above list. In some embodiments, in combinations 1-32 of the above cases, CY5 and/or Atto647N is replaced with GM656 or Alexa Fluor647. In some embodiments, in combinations 1-32 of the above cases, Atto532 and/or Alexa Fluor532 is replaced with Atto Rho6G or CY3. In some embodiments, in combinations 1-32 of the above cases, CY5 and/or Atto647N is replaced with GM656 or Alexa Fluor647, and Atto532 and/or Alexa Fluor532 is replaced with Atto Rho6G or CY3.

In some other embodiments, the fluorophores carried by the four types of reversible terminators may be a combination of a blue fluorophore and a green fluorophore. For example, in combinations 1-32 of the above cases, CY5 and/or Atto647N is replaced with one type of blue-emitting fluorescent dyes.

Based on the above embodiments, the first cleavable group may further be introduced into the reversible terminator including the second cleavable group, such that after both the first cleavable group and the second cleavable group are cleaved, the four types of nucleotides or the derivatives thereof have the same cleavage scars on the base side, and thus the biochemical reaction and signal identification involved in the sequencing are more stable.

As another implementation of the above embodiments, the structures of the four types of reversible terminators are as follows:
the structure of the first reversible terminator is: a first nucleotide or a derivative thereof-the first cleavable group-the first fluorophore;
the structure of the second reversible terminator is: a second nucleotide or a derivative thereof-the first cleavable group-the second cleavable group-the first fluorophore;
the structure of the third reversible terminator is: a third nucleotide or a derivative thereof-the first cleavable group-the second cleavable group-the second fluorophore;
the structure of the fourth reversible terminator is: a fourth nucleotide or a derivative thereof-the first cleavable group-the second fluorophore,
where the first nucleotide or the derivative thereof, the second nucleotide or the derivative thereof, the third nucleotide or the derivative thereof, and the fourth nucleotide or the derivative thereof are independently selected from one of adenine or a derivative thereof, thymine or a derivative thereof, cytosine guanine or a derivative thereof, and guanine or a derivative thereof, and the first nucleotide or the derivative thereof, the second nucleotide or the derivative thereof, the third nucleotide or the derivative thereof, and the fourth nucleotide or the derivative thereof are different from one another.

Similarly, in the embodiments of the present application, the combination of the first cleavable group and the second cleavable group satisfies that: under the same cleavage condition, when the second cleavable group is cleaved, the first cleavable group is not affected and still remains in the reversible terminator structure. In some embodiments, the first cleavable group in the above four types of reversible terminators is —N3-, and the second cleavable group is —SS—. Certainly, the combination of the first cleavable group and the second cleavable group is not limited thereto.

Illustratively, the four types of reversible terminators include at least one of the following combination 33 to combination 48; or the four types of reversible terminators include at least one of the following combination 49 to combination 64:
combination 33: adenine or a derivative thereof-N3-Atto647N; thymine or a derivative thereof-N3-SS-Atto532; cytosine or a derivative thereof-N3-Alexa Fluor532; and guanine or a derivative thereof-N3-SS-CY5;
combination 34: adenine or a derivative thereof-N3-Atto647N; thymine or a derivative thereof-N3-SS-Atto532; cytosine or a derivative thereof-N3-Alexa Fluor532; and guanine or a derivative thereof-N3-SS-Atto647N;
combination 35: adenine or a derivative thereof-N3-Atto647N; thymine or a derivative thereof-N3-SS-Alexa Fluor532; cytosine or a derivative thereof-N3-Atto532; and guanine or a derivative thereof-N3-SS-Atto647N;
combination 36: adenine or a derivative thereof-N3-CY5; thymine or a derivative thereof-N3-SS-Atto532; cytosine or a derivative thereof-N3-Alexa Fluor532; and guanine or a derivative thereof-N3-SS-CY5;
combination 37: adenine or a derivative thereof-N3-CY5; thymine or a derivative thereof-N3-SS-Alexa Fluor532; cytosine or a derivative thereof-N3-Atto532; and guanine or a derivative thereof-N3-SS-CY5;
combination 38: adenine or a derivative thereof-N3-CY5; thymine or a derivative thereof-N3-SS-Atto532; cytosine or a derivative thereof-N3-Atto532; and guanine or a derivative thereof-N3-SS-CY5;
combination 39: adenine or a derivative thereof-N3-CY5; thymine or a derivative thereof-N3-SS-Alexa Fluor532; cytosine or a derivative thereof-N3-Alexa Fluor532; and guanine or a derivative thereof-N3-SS-CY5;
combination 40: adenine or a derivative thereof-N3-Atto647N; thymine or a derivative thereof-N3-SS-Atto532; cytosine or a derivative thereof-N3-Atto532; and guanine or a derivative thereof-N3-SS-CY5;
combination 41: adenine or a derivative thereof-N3-Atto647N; thymine or a derivative thereof-N3-SS-Alexa Fluor532; cytosine or a derivative thereof-N3-Atto532; and guanine or a derivative thereof-N3-SS-CY5;
combination 42: adenine or a derivative thereof-N3-Atto647N; thymine or a derivative thereof-N3-SS-Alexa Fluor532; cytosine or a derivative thereof-N3-Alexa Fluor532; and guanine or a derivative thereof-N3-SS-CY5;
combination 43: adenine or a derivative thereof-N3-CY5; thymine or a derivative thereof-N3-SS-Alexa Fluor532; cytosine or a derivative thereof-N3-Alexa Fluor532; and guanine or a derivative thereof-N3-SS-Atto647N;
combination 44: adenine or a derivative thereof-N3-CY5; thymine or a derivative thereof-N3-SS-Atto532; cytosine or a derivative thereof-N3-Atto532; and guanine or a derivative thereof-N3-SS-Atto647N;
combination 45: adenine or a derivative thereof-N3-CY5; thymine or a derivative thereof-N3-SS-Atto532; cytosine or a derivative thereof-N3-Alexa Fluor532; and guanine or a derivative thereof-N3-SS-Atto647N;
combination 46: adenine or a derivative thereof-N3-CY5; thymine or a derivative thereof-N3-SS-Alexa Fluor532; cytosine or a derivative thereof-N3-Atto532; and guanine or a derivative thereof-N3-SS-Atto647N;
combination 47: adenine or a derivative thereof-N3-Atto647N; thymine or a derivative thereof-N3-SS-Alexa Fluor532; cytosine or a derivative thereof-N3-Alexa Fluor532; and guanine or a derivative thereof-N3-SS-Atto647N;
combination 48: adenine or a derivative thereof-N3-Atto647N; thymine or a derivative thereof-N3-SS-Atto532; cytosine or a derivative thereof-N3-Atto532; and guanine or a derivative thereof-N3-SS-Atto647N;
combination 49: adenine or a derivative thereof-N3-Atto532; thymine or a derivative thereof-N3-SS-Atto647N; cytosine or a derivative thereof-N3-Atto647N; and guanine or a derivative thereof-N3-SS-Atto532;
combination 50: adenine or a derivative thereof-N3-Alexa Fluor532; thymine or a derivative thereof-N3-SS-Atto647N; cytosine or a derivative thereof-N3-Atto647N; and guanine or a derivative thereof-N3-SS-Atto532;
combination 51: adenine or a derivative thereof-N3-Atto532; thymine or a derivative thereof-N3-SS-Atto647N; cytosine or a derivative thereof-N3-Atto647N; and guanine or a derivative thereof-N3-SS-Alexa Fluor532;
combination 52: adenine or a derivative thereof-N3-Alexa Fluor532; thymine or a derivative thereof-N3-SS-Atto647N; cytosine or a derivative thereof-N3-Atto647N; and guanine or a derivative thereof-N3-SS-Alexa Fluor532;
combination 53: adenine or a derivative thereof-N3-Atto532; thymine or a derivative thereof-N3-SS-CY5; cytosine or a derivative thereof-N3-Atto647N; and guanine or a derivative thereof-N3-SS-Atto532;
combination 54: adenine or a derivative thereof-N3-Alexa Fluor532; thymine or a derivative thereof-N3-SS-CY5; cytosine or a derivative thereof-N3-Atto647N; and guanine or a derivative thereof-N3-SS-Atto532;
combination 55: adenine or a derivative thereof-N3-Atto532; thymine or a derivative thereof-N3-SS-CY5; cytosine or a derivative thereof-N3-Atto647N; and guanine or a derivative thereof-N3-SS-Alexa Fluor532;
combination 56: adenine or a derivative thereof-N3-Alexa Fluor532; thymine or a derivative thereof-N3-SS-CY5; cytosine or a derivative thereof-N3-Atto647N; and guanine or a derivative thereof-N3-SS-Alexa Fluor532;
combination 57: adenine or a derivative thereof-N3-Atto532; thymine or a derivative thereof-N3-SS-Atto647N; cytosine or a derivative thereof-N3-CY5; and guanine or a derivative thereof-N3-SS-Atto532;
combination 58: adenine or a derivative thereof-N3-Alexa Fluor532; thymine or a derivative thereof-N3-SS-Atto647N; cytosine or a derivative thereof-N3-CY5; and guanine or a derivative thereof-N3-SS-Atto532;
combination 59: adenine or a derivative thereof-N3-Atto532; thymine or a derivative thereof-N3-SS-Atto647N; cytosine or a derivative thereof-N3-CY5; and guanine or a derivative thereof-N3-SS-Alexa Fluor532;
combination 60: adenine or a derivative thereof-N3-Alexa Fluor532; thymine or a derivative thereof-N3-SS-Atto647N; cytosine or a derivative thereof-N3-CY5; and guanine or a derivative thereof-N3-SS-Alexa Fluor532;
combination 61: adenine or a derivative thereof-N3-Alexa Fluor532; thymine or a derivative thereof-N3-SS-CY5; cytosine or a derivative thereof-N3-CY5; and guanine or a derivative thereof-N3-SS-Atto532;
combination 62: adenine or a derivative thereof-N3-Atto532; thymine or a derivative thereof-N3-SS-CY5; cytosine or a derivative thereof-N3-CY5; and guanine or a derivative thereof-N3-SS-Alexa Fluor532;
combination 63: adenine or a derivative thereof-N3-Atto532; thymine or a derivative thereof-N3-SS-CY5; cytosine or a derivative thereof-N3-CY5; and guanine or a derivative thereof-N3-SS-Atto532; and
combination 64: adenine or a derivative thereof-N3-Alexa Fluor532; and thymine or a derivative thereof-N3-SS-CY5.

In some embodiments, in the above combinations 33-64, a double cleavable group-N3-SS— linked to the purine or the derivative thereof or the pyrimidine or the derivative thereof is replaced with a single cleavable group-N3-, and a single cleavable group-N3- linked to the purine or the derivative thereof or the pyrimidine or the derivative thereof is replaced with a double cleavable group-N3-SS—. In this case, due to the weak reducibility of —SS—, the first fluorophore and the second fluorophore in two types of the reversible terminators having the cleavable group-N3-SS-in the structure may be preferentially cleaved along with the cleavage of —SS—, while-N3- is retained. In this case, the four types of reversible terminators all have the first cleavable group-N3-. Identification of the four types of reversible terminators can be achieved by comparing changes in fluorescence signal before and after cleavage of —SS—. After cleavage of —N3- of the four types of reversible terminators, nucleotides or derivatives thereof having the same or similar cleavage scars can be obtained.

Similarly, in some embodiments, in the above combinations 33-64, a single cleavable group-N3- linked to adenine or the derivative thereof is replaced with a double cleavable group-N3-SS—, and a double cleavable group-N3-SS— linked to guanine or the derivative thereof is replaced with a single cleavable group-N3-. In some embodiments, in the above combinations 33-64, a double cleavable group-N3-SS— linked to thymine or a derivative thereof is replaced with a single cleavable group-N3-, and a single cleavable group-N3- linked to cytosine or a derivative thereof is replaced with a double cleavable group-N3-SS—.

The above combinations 33-64 provide a plurality of reversible terminator combinations that include red fluorophores and green fluorophores. Certainly, the combinations of red fluorophores and green fluorophores are not limited to the above list. In some embodiments, in combinations 33-64 of the above cases, CY5 and/or Atto647N is replaced with GM656 or Alexa Fluor647. In some embodiments, in combinations 33-64 of the above cases, Atto532 and/or Alexa Fluor532 is replaced with Atto Rho6G or CY3. In some embodiments, in combinations 33-64 of the above cases, CY5 and/or Atto647N is replaced with GM656 or Alexa Fluor647, and Atto532 and/or Alexa Fluor532 is replaced with Atto Rho6G or CY3.

In some other embodiments, the fluorophores carried by the four types of reversible terminators may be a combination of a blue fluorophore and a green fluorophore. For example, in combinations 33-64 of the above cases, CY5 and/or Atto647N is replaced with one type of blue-emitting fluorescent dyes.

In some embodiments, the final concentration of each reversible terminator is 0.05 nmol/Lol/L-2000 nmol/Lol/L. It should be understood that the final concentration or working concentration of the reversible terminator indicated in the embodiments of the present application refers to the concentration of the reversible terminator when a solution containing the reversible terminator is contacted with the nucleic acid template-primer complex. Illustratively, when a reaction solution containing the reversible terminator is introduced onto the solid carrier, the concentration of the reversible terminator in the reaction solution is the final concentration of the reversible terminator. In one embodiment, this step involves pumping different reagents from different kits, mixing the reagents to form a mixed solution, and allowing the mixed solution to flow onto the surface of the solid carrier, and in this case, the final concentration of the reversible terminator should be the concentration of the reversible terminator in the mixed solution. In the embodiments of the present application, the final concentration of the reversible terminator is set to be adjusted within the above range under polymerase chain reaction conditions, which is beneficial for reducing the adsorption of the reversible terminator in other regions except the sites on the surface of the solid carrier, thus reducing non-specific adsorption formed thereby. The fluorophores in the reversible terminators at these non-specific adsorption positions are excited in step 3 to generate optical signals, which increases the background noise in the images obtained by imaging and interferes with the identification of the optical signals of the fluorophores at the sites, thereby affecting the accuracy of base calling.

In the embodiments of the present application, the final concentration of each reversible terminator may be adjusted within the range of 0.05 nmol/Lol/L-2000 nmol/Lol/L based on the type of the reversible terminator, the concentration of the polymerase in the reaction system, and the like.

As a first implementation, the nucleotide or the derivative thereof in the reversible terminator includes an adenine nucleotide or a derivative thereof. In an exemplary embodiment of the present disclosure, the structure of the corresponding reversible terminator is dA-N3-Atto647N, and when dA-N3-Atto647N is included in the reaction system of the polymerase chain reaction in step (2), the final concentration of dA-N3-Atto647N is 10 nmol/L-700 nmol/L, specifically 10 nmol/L, 15 nmol/L, 20 nmol/L, 30 nmol/L, 40 nmol/L, 50 nmol/L, 60 nmol/L, 70 nmol/L, 80 nmol/L, 90 nmol/L, 100 nmol/L, 125 nmol/L, 150 nmol/L, 175 nmol/L, 200 nmol/L, 225 nmol/L, 250 nmol/L, 275 nmol/L, 300 nmol/L, 325 nmol/L, 350 nmol/L, 375 nmol/L, 400 nmol/L, 425 nmol/L, 450 nmol/L, 475 nmol/L, 500 nmol/L, 525 nmol/L, 550 nmol/L, 575 nmol/L, 600 nmol/L, 625 nmol/L, 650 nmol/L, 675 nmol/L, 700 nmol/L, or the like.

As a second implementation, the nucleotide or the derivative thereof in the reversible terminator includes a guanine nucleotide or a derivative thereof. In an exemplary embodiment of the present disclosure, the structure of the corresponding reversible terminator is dG-SS-Atto647N or dG-N3-SS-CY5, and when dG-SS-Atto647N or dG-N3-SS-CY5 is included in the reaction system of the polymerase chain reaction in step (2), the final concentration of dG-SS-Atto647N or dG-N3-SS-CY5 is 0.05 nmol/L-5 nmol/L, specifically, 0.05 nmol/L, 0.06 nmol/L, 0.07 nmol/L, 0.08 nmol/L, 0.09 nmol/L, 0.1 nmol/L, 0.15 nmol/L, 0.25 nmol/L, 0.35 nmol/L, 0.45 nmol/L, 0.55 nmol/L, 0.65 nmol/L, 0.75 nmol/L, 0.85 nmol/L, 0.95 nmol/L, 1 nmol, 1.15 nmol/L, 1.25 nmol/L, 1.35 nmol/L, 1.45 nmol/L, 1.55 nmol/L, 1.65 nmol/L, 1.75 nmol/L, 1.85 nmol/L, 1.95 nmol/L, 2 nmol, 2.15 nmol/L, 2.25 nmol/L, 2.35 nmol/L, 2.45 nmol/L, 2.55 nmol/L, 2.65 nmol/L, 2.75 nmol/L, 2.85 nmol/L, 2.95 nmol/L, 3 nmol, 3.15 nmol/L, 3.25 nmol/L, 3.35 nmol/L, 3.45 nmol/L, 3.55 nmol/L, 3.65 nmol/L, 3.75 nmol/L, 3.85 nmol/L, 3.95 nmol/L, 4 nmol, 4.15 nmol/L, 4.25 nmol/L, 4.35 nmol/L, 4.45 nmol/L, 4.55 nmol/L, 4.65 nmol/L, 4.75 nmol/L, 4.85 nmol/L, 4.95 nmol/L, 5 nmol, or the like.

As a third implementation, the nucleotide or the derivative thereof in the reversible terminator includes a cytosine nucleotide or a derivative thereof. In an exemplary embodiment of the present disclosure, the structure of the corresponding reversible terminator is dC-N3-Atto532 or dC-N3-Alexa Fluor532, and when dC-N3-Atto532 or dC-N3-Alexa Fluor532 is included in the reaction system of the polymerase chain reaction in step (2), the final concentration of dC-N3-Atto532 or dC-N3-Alexa Fluor532 is 10 nmol/L-700 nmol/L, specifically 10 nmol/L, 15 nmol/L, 20 nmol/L, 30 nmol/L, 40 nmol/L, 50 nmol/L, 60 nmol/L, 70 nmol/L, 80 nmol/L, 90 nmol/L, 100 nmol/L, 125 nmol/L, 150 nmol/L, 175 nmol/L, 200 nmol/L, 225 nmol/L, 250 nmol/L, 275 nmol/L, 300 nmol/L, 325 nmol/L, 350 nmol/L, 375 nmol/L, 400 nmol/L, 425 nmol/L, 450 nmol/L, 475 nmol/L, 500 nmol/L, 525 nmol/L, 550 nmol/L, 575 nmol/L, 600 nmol/L, 625 nmol/L, 650 nmol/L, 675 nmol/L, 700 nmol/L, or the like.

As a fourth implementation, the nucleotide or the derivative thereof in the reversible terminator includes a thymine nucleotide or a derivative thereof or a uracil nucleotide or a derivative thereof. In an exemplary embodiment of the present disclosure, the structure of the corresponding reversible terminator is dT-SS-Atto532 or dT-N3-SS-Atto532, and when dT-SS-Atto532 or dT-N3-SS-Atto532 is included in the reaction system of the polymerase chain reaction in step (2), the final concentration of dT-SS-Atto532 or dT-N3-SS-Atto532 is 5 nmol/L-80 nmol/L, specifically, 5 nmol/L, 6 nmol/L, 7 nmol/L, 8 nmol/L, 9 nmol/L, 10 nmol/L, 12 nmol/L, 14 nmol/L, 16 nmol/L, 18 nmol/L, 20 nmol/L, 22 nmol/L, 24 nmol/L, 26 nmol/L, 28 nmol/L, 30 nmol/L, 32 nmol/L, 34 nmol/L, 36 nmol/L, 38 nmol/L, 40 nmol/L, 42 nmol/L, 44 nmol/L, 46 nmol/L, 48 nmol/L, 50 nmol/L, 52 nmol/L, 54 nmol/L, 56 nmol/L, 58 nmol/L, 60 nmol/L, 62 nmol/L, 64 nmol/L, 66 nmol/L, 68 nmol/L, 70 nmol/L, 72 nmol/L, 74 nmol/L, 76 nmol/L, 78 nmol/L, 80 nmol/L, or the like.

In the embodiments of the present application, the polymerase used for causing the reversible terminators to undergo a polymerization reaction may be a DNA polymerase, an RNA polymerase, or may contain both a DNA polymerase and an RNA polymerase. In some embodiments, the polymerase is a DNA polymerase, including but not limited to, one or more of 9°N enzyme, Pfu, KOD1, and mmol/LS2.

The final concentration of the polymerase indicated in the embodiments of the present application refers to the concentration of the polymerase when a solution containing the polymerase is contacted with the solid carrier. Illustratively, when a reaction solution containing the polymerase is introduced onto the solid carrier, the concentration of the polymerase in the reaction solution is the final concentration of the polymerase. In one embodiment, this step involves pumping different reagents from different kits, mixing the reagents to form a mixed solution, and allowing the mixed solution to flow onto the surface of the solid carrier, and in this case, the final concentration of the polymerase should be the concentration of the polymerase in the mixed solution. The final concentration of the polymerase is set to be 0.005 mg/mL-0.02 mg/mL in the embodiments of the present application, such that under polymerase chain reaction conditions, the adsorption of the reversible terminator in other regions except the sites on the surface of the solid carrier is reduced, thus reducing non-specific adsorption formed thereby. The fluorophores in the reversible terminators at these non-specific adsorption positions are excited in step 3 and step 5 to generate optical signals, which increases the background noise in the images obtained by imaging and interferes with the identification of the optical signals at the sites, thereby affecting the accuracy of base calling.

Illustratively, the final concentration of the polymerase is 0.005 mg/mL, 0.006 mg/mL, 0.007 mg/mL, 0.008 mg/mL, 0.009 mg/mL, 0.01 mg/mL, 0.011 mg/mL, 0.012 mg/mL, 0.013 mg/mL, 0.014 mg/mL, 0.015 mg/mL, 0.016 mg/mL, 0.017 mg/mL, 0.018 mg/mL, 0.019 mg/mL, 0.02 mg/mL, or the like.

In some embodiments, the polymerase chain reaction is performed at a pH of 8.0-9.0. Therefore, the concentration of the reversible terminators participating in the polymerase chain reaction, the concentration of the polymerase, and the pH condition of the reaction system may be regulated and controlled to effectively reduce the adsorption of the reversible terminators in other regions except the sites on the surface of the solid carrier, thus reducing non-specific adsorption formed thereby, reducing the background noise in the images obtained in step 3 and step 5, and improving the accuracy of base calling.

In the embodiments of the present application, the reaction system of the polymerase chain reaction further includes a solvent for dissolving the reversible terminators and the polymerase, and the solvent may be a buffer such as tris(hydroxymethyl)aminomethane buffer, glycine, ethanolamine, tetraethylethylenediamine, tetramethylethylenediamine, N-butyldiethanolamine, diethylaminoethanol, Hepes buffer, or N,N-dihydroxyethylglycine.

In some embodiments, the concentration of the buffer is 0.01 mol/L-2 mol/L. The pH environment provided by the buffer at a concentration of 0.01 mol/L-2 mol/L is beneficial for improving the uniformity and stability of the reduction reaction. Illustratively, the concentration of the buffer in the mixed solution is 10 mmol/L, 15 mmol/L, 20 mmol/L, 25 mmol/L, 30 mmol/L, 35 mmol/L, 40 mmol/L, 45 mmol/L, 50 mmol/L, 55 mmol/L, 60 mmol/L, 65 mmol/L, 70 mmol/L, 75 mmol/L, 80 mmol/L, 85 mmol/L, 90 mmol/L, 95 mmol/L, 100 mmol/L, 105 mmol/L, 110 mmol/L, 115 mmol/L, 120 mmol/L, 125 mmol/L, 130 mmol/L, 135 mmol/L, 140 mmol/L, 145 mmol/L, 150 mmol/L, 155 mmol/L, 160 mmol/L, 165 mmol/L, 170 mmol/L, 175 mmol/L, 180 mmol/L, 0.2 mol/L, 0.3 mol/L, 0.4 mol/L, 0.5 mol/L, 0.6 mol/L, 0.7 mol/L, 0.8 mol/L, 0.9 mol/L, 1.0 mol/L, 1.1 mol/L, 1.2 mol/L, 1.3 mol/L, 1.4 mol/L, 1.5 mol/L, 1.6 mol/L, 1.7 mol/L, 1.8 mol/L, 1.9 mol/L, 2.0 mol/L, or the like.

The binding of the four types of reversible terminators to the nucleic acid template-primer complex is achieved by polymerase chain reaction, and in this case, the solid carrier carries one type of fluorophore at each site to which the nucleic acid template-primer is bound. Reversible terminators that are not bound to the nucleic acid template-primer complex are removed by washing. In this case, since the fluorophores and the cleavable groups of the reversible terminators added simultaneously are different, the specific reversible terminator incorporated into the extension product obtained in step (2) can be distinguished by performing subsequent step (3) to step (5).

In step (3), fluorescence signals of the extension product are acquired to obtain image 1, where image 1 includes a first signal, a second signal, a third signal, and a fourth signal from the first reversible terminator, the second reversible terminator, the third reversible terminator, and the fourth reversible terminator, respectively.

Specifically, excitation light excites the fluorophores in the reversible terminators to generate fluorescence signals. The first signal, the second signal, the third signal, and the fourth signal from the first reversible terminator, the second reversible terminator, the third reversible terminator, and the fourth reversible terminator are acquired.

It should be understood that different fluorophores require excitation light of different wavelengths to generate optical signals. In some embodiments, the fluorophores of the reversible terminators added in step (2) at least include the first fluorophore and the second fluorophore, and the excitation wavelengths of the first fluorophore and the second fluorophore differ by at least 50 nmol/L, so as to increase the difference in optical signals generated by the first fluorophore and the second fluorophore. The greater the difference between the excitation wavelengths of the first fluorophore and the second fluorophore, the more distinct the difference between the optical signals generated by the first fluorophore and the second fluorophore, and the easier the two fluorophores are to be identified.

In this step, after the fluorophores are excited to generate fluorescence signals, exposure processing is performed. In some embodiments, the exposure processing lasts for 100 ms-400 ms. By controlling the exposure time, the difference in the intensity of the optical signals generated by the fluorophores may be increased to a certain extent, thereby facilitating the identification of the types of reversible terminators introduced at the sites and improving the accuracy of base calling. Illustratively, the exposure processing lasts for 100 ms, 120 ms, 150 ms, 180 ms, 200 ms, 220 ms, 250 ms, 280 ms, 300 ms, 320 ms, 350 ms, 380 ms, 400 ms, or the like. It should be understood that the time of the exposure processing may be adjusted based on the type of the fluorophore, so as to achieve the purpose of increasing the difference in the intensity of the optical signals generated by the fluorophores. In one embodiment, the first fluorophore is a red-light-excitable fluorophore, and the exposure time for the red-light-excitable fluorophore is 100 ms-400 ms, such as 100 ms, 120 ms, 150 ms, 180 ms, 200 ms, 220 ms, 250 ms, 280 ms, 300 ms, 320 ms, 350 ms, 380 ms, or 400 ms; the second fluorophore is a green-light-excitable fluorophore, and the exposure time of the green-light-excitable fluorophore is 50 ms-100 ms, such as 50 ms, 60 ms, 70 ms, 80 ms, 90 ms, or 100 ms.

After the fluorophores are excited to generate optical signals, the extension product is imaged to obtain image 1.

In the embodiments of the present application, four types of reversible terminators are designed, such that signals of any two types of the reversible terminators can be different from signals of the other two types of the reversible terminators. For example, in the embodiments of the present application, the first signal and the second signal are different from the third signal and the fourth signal. In some embodiments, the first signal and the second signal are the same and are different in color from the third signal and the fourth signal, and the third signal and the fourth signal are the same. In this case, based on signal differences in image 1, the first reversible terminator and the second reversible terminator can be distinguished from the third reversible terminator and the fourth reversible terminator. In the embodiments of the present application, whether signals are identical or different may be characterized by signal features, and the signal features include fluorescence color. Illustratively, the first signal and the second signal are red fluorescence signals, and the third signal and the fourth signal are green fluorescence signals. Certainly, the opposite configuration is also possible, and the example is not limited thereto.

In step (4), the second cleavable group in the extension product is cleaved to obtain a reaction product.

In this step, an appropriate cleavage method is selected to cleave the second cleavable group based on the binding properties of the second cleavable group in the reversible terminator. Suitable cleavage methods include, but are not limited to, chemical cleavage, enzymatic cleavage, and photocleavage. It should be noted that in this step, the condition for cleaving the second cleavable group in the extension product does not involve cleaving the first cleavable group in the reversible terminator. Therefore, through this step, the fluorophore in the reversible terminator having the second cleavable group is cleaved from the reversible terminator together with the second cleavable group. Since the first cleavable group in the reversible terminator having the first cleavable group is retained, the fluorophores in these reversible terminators are also retained.

In some embodiments, the second cleavable group is a group including a disulfide bond, and the first cleavable group is selected from at least one of —N3-, —CH₂F, —C(O)NHBu, —CHF₂, —C(O)OH, —C₆, and/or —ArC₆. In this case, the second cleavable group in the reversible terminator is cleaved by adding a cleavage reagent including a reducing agent, and the first cleavable group in the reversible terminator is retained.

Illustratively, the reducing agent for cleaving the second cleavable group includes, but is not limited to, any one or more of DTT, glutathione, and β-mercaptoethanol.

In some embodiments, the concentration of the reducing agent is 10 mmol/L-100 mmol/L; specifically, the concentration of the reducing agent may be 10 mmol/L, 15 mmol/L, 20 mmol/L, 25 mmol/L, 30 mmol/L, 35 mmol/L, 40 mmol/L, 45 mmol/L, 50 mmol/L, 55 mmol/L, 60 mmol/L, 65 mmol/L, 70 mmol/L, 75 mmol/L, 80 mmol/L, 85 mmol/L, 90 mmol/L, 95 mmol/L, 100 mmol/L, or the like.

In one embodiment, the second cleavable group is cleaved using glutathione as the reducing agent, and the working concentration of glutathione is 10 mmol/L-100 mmol/L. Illustratively, the concentration of glutathione may be 10 mmol/L, 15 mmol/L, 20 mmol/L, 25 mmol/L, 30 mmol/L, 35 mmol/L, 40 mmol/L, 45 mmol/L, 50 mmol/L, 55 mmol/L, 60 mmol/L, 65 mmol/L, 70 mmol/L, 75 mmol/L, 80 mmol/L, 85 mmol/L, 90 mmol/L, 95 mmol/L, 100 mmol/L, or the like.

In one embodiment, the reducing agent cleavage reagent includes: 0.2 mol/L-2 mol/L Tris-Base, 0.2 mol/L-2 mol/L sodium chloride, 10 mmol/L-100 mmol/L GSH, and 0.01%-0.1% Tween 20, at a pH of 8.0-10. Illustratively, the reducing agent cleavage reagent is: 1 mol/L Tris-Base, 0.5 mol/L sodium chloride, 50 mmol/L GSH, and 0.05% Tween 20, at a pH of 9.0.

Thereafter, the extension product is washed to remove the cleaved fluorophores and the cleavage reagent.

In step (5), fluorescence signals of the reaction product are acquired to obtain image 2, where image 2 includes a fifth signal, a sixth signal, a seventh signal, and an eighth signal from the first reversible terminator, the second reversible terminator, the third reversible terminator, and the fourth reversible terminator, respectively.

Specifically, the fifth signal, the sixth signal, the seventh signal, and the eighth signal from the first reversible terminator, the second reversible terminator, the third reversible terminator, and the fourth reversible terminator, respectively, are acquired again, and the corresponding image 2 is obtained. In some embodiments, for the conditions for acquiring the fluorescence signals in this step, reference may be made to step (3).

In this step, since the second cleavable group together with the fluorophore is cleaved from the reversible terminator, the first cleavable group and the fluorophore on the reversible terminator where it is located are retained. Therefore, when capturing an image, no fluorescence signal can be acquired at this time from reversible terminators having the second cleavable group, while fluorescence signals can still be acquired from reversible terminators having the first cleavable group. In image 2 thus obtained, the reversible terminators including the second cleavable group no longer display fluorescence signals; while the reversible terminators including the second cleavable group retain the same fluorescence signals as those in image 1.

Since the fluorescence colors of the fluorophores of reversible terminators having the same cleavable group are different, or the fluorescence colors of the fluorophores of reversible terminators having different cleavable groups are the same, the reversible terminators with the same fluorescence color can be further distinguished by the cleavage in this step.

Specifically, in image 1, the fluorescence colors of the first signal and the second signal are the same, the fluorescence colors of the third signal and the fourth signal are the same, and the fluorescence colors of the first signal and the second signal are different from the fluorescence colors of the third signal and the fourth signal. Therefore, the first reversible terminator and the second reversible terminator can be distinguished from the third reversible terminator and the fourth reversible terminator. In image 2, the sixth signal is different from the second signal, and the seventh signal is different from the third signal. In this case, the first reversible terminator can be distinguished from the second reversible terminator, and the third reversible terminator can be distinguished from the fourth reversible terminator. By combining changes in optical signals in image 1 and image 2, the types of reversible terminators bound to the nucleic acid template-primer complex can be ultimately determined.

Thereafter, the first cleavable group in the extension product may be cleaved. In this step, an appropriate cleavage method is selected to cleave the first cleavable group based on the binding properties of the first cleavable group in the reversible terminator. Suitable cleavage methods include, but are not limited to, chemical cleavage, enzymatic cleavage, and photocleavage. Through cleavage in this step, the nucleotide analog incorporated at the terminal end of the primer strand of the extension product no longer has a fluorophore and a cleavable group, and the terminal end of the primer strand of the extension product restores activity for polymerization of the next reversible terminator.

In some embodiments, the first cleavable group is selected from at least one of —N3-, —CH₂F, —C(O)NHBu, —CHF₂, —C(O)OH, —C₆, and/or —ArC₆. Illustratively, the first cleavable group is —N3-, and in this case, the first cleavable group in the reversible terminator is cleaved by adding an organic phosphine cleavage reagent. In some embodiments, the concentration of organic phosphine is 5 mmol-20 mmol/L; illustratively, the concentration of organic phosphine may be 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, 15 mmol/L, 16 mmol/L, 17 mmol/L, 18 mmol/L, 19 mmol/L, 20 mmol/L, or the like.

Illustratively, the organic phosphine cleavage reagent for cleaving the first cleavable group includes, but is not limited to, any one or more of tris(3-hydroxypropyl)phosphine, tris(2-carboxyethyl)phosphine, tris(hydroxymethyl)phosphine, triphenylphosphine, or tris(2-hydroxyethyl)phosphine. In one embodiment, tris(3-hydroxypropyl)phosphine is used as the cleavage reagent to cleave the second cleavable group, and the working concentration of tris(3-hydroxypropyl)phosphine is 5 mmol/L-20 mmol/L; illustratively, the concentration of organic phosphine may be 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, 15 mmol/L, 16 mmol/L, 17 mmol/L, 18 mmol/L, 19 mmol/L, 20 mmol/L, or the like.

In some embodiments, the organic phosphine cleavage reagent includes 0.2 mol/L-2 mol/L Tris-Base, 0.2 mol/L-2 mol/L sodium chloride, 10 mmol/L-100 mmol/L THPP, and 0.01%-0.1% Tween 20, at a pH of 8-10. Illustratively, the tris(3-hydroxypropyl)phosphine cleavage reagent is 1 mol/L Tris-Base, 0.5 mol/L sodium chloride, 10 mmol/L THPP, and 0.05% Tween 20, at a pH of 9.0.

In some embodiments, when the structure of the reversible terminator includes only the second cleavable group, the method further includes performing blocking on the cleavage site after any one of or between the above step (3) to step (7).

In the embodiments of the present application, step (2) to step (7) may be repeated one or more times, and each repetition achieves binding of one of the four types of reversible terminators to the nucleic acid template-primer complex, thereby completing one single-base extension, and identifying the base type thereof. Accordingly, by repeating the process multiple times, two-color sequencing is achieved to obtain the base sequence of the template strand.

### Example 1

### Adsorption Test for Sequencing:

An adsorption test of reversible terminator concentrations was performed to determine appropriate concentrations of the four nucleotides and polymerase during the base extension reaction.

The method for the adsorption test was as follows:

A surface-processed solid carrier including a plurality of sites was provided, and each of the sites was capable of immobilizing a nucleic acid template-primer complex, but without immobilizing the nucleic acid template-primer complex itself.

Nucleotide solutions of dA-N3-Atto647N, dT-N3-SS-Atto532, dC-N3-Alexa Fluor532, and dG-N3-SS-CY5 were prepared, respectively, and 9°N enzyme was added to each nucleotide solution to form extension reagents, which were then separately introduced onto the surface of the solid carrier. For convenience of description, the extension reagent containing dA-N3-Atto647N and 9°N enzyme was named as dA-N3-Atto647N extension reagent, the extension reagent containing dT-N3-SS-Atto532 and 9°N enzyme was named as dT-N3-SS-Atto532 extension reagent, the extension reagent containing dC-N3-Alexa Fluor532 and 9°N enzyme was named as dC-N3-Alexa Fluor532 extension reagent, and the extension reagent containing dG-N3-SS-CY5 and 9°N enzyme was named as dG-N3-SS-CY5 extension reagent. Different nucleotide concentrations and 9°N enzyme concentrations were set in the extension reagents.

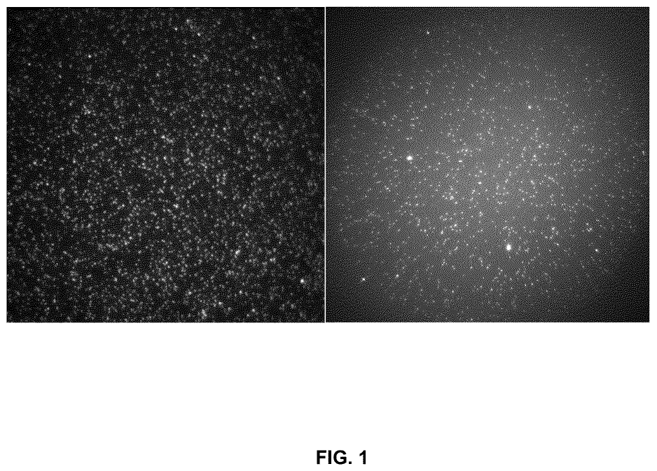

Excitation light having a wavelength consistent with the fluorophore carried by the extension reagent to be added was used to excite the fluorophore to generate fluorescence signals, and the solid carrier after excitation processing was imaged to obtain an image. The results are shown in FIG. 1.

Data analysis was performed for the image, and the average number of fluorescence signal points (number of adsorption points) in the image was counted. Based on the statistical results, the solubility of the nucleotide solution and the polymerase in the subsequent examples was set as follows: dA-N3-Atto647N at 10 nmol/L-500 nmol/L, dC-N3-Alexa Fluor532 at 100 nmol/L-700 nmol/L, dT-N3-SS-Atto532 at 20 nmol/L-80 nmol/L, dG-N3-SS-CY5 at 0.1 nmol/L-5 nmol/L, and the concentration of 9°N was 0.01 mg/mL-0.1 mg/mL.

### Example 2

Test for efficiency of disulfide bond cleavage by glutathione, including the following steps:

A nucleotide solution of ddG-SS-Atto647N was prepared, and 9°N enzyme was added to each nucleotide solution to form an extension reagent. In the extension reagent, the concentration of the 9°N enzyme was set to 0.02 mg/mL; and the concentration of ddG-SS-Atto647N was 5 nmol/L.

A synthesis reagent was introduced onto the surface of the solid carrier at a temperature of 60° C. for reaction for 2 min. After the reaction was completed, the surface of the solid carrier was washed.

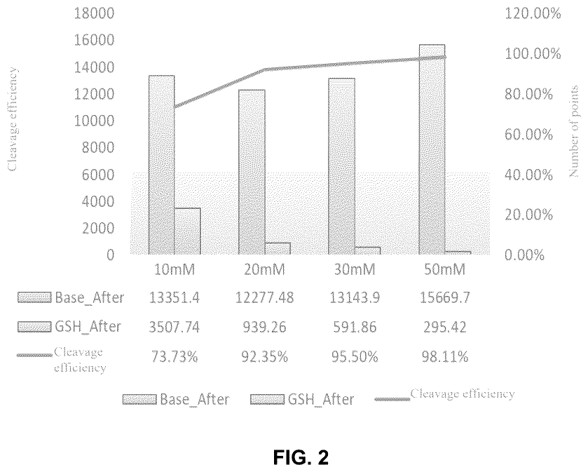

The surface of the solid carrier was irradiated with excitation light from a 640 nm laser and the fluorophores on the extension reagent were excited to generate optical signals, and the fluorescence signals of the solid carrier were acquired to obtain image 1. Subsequently, reaction was allowed at 37° C. for 2 min using a GSH cleavage reagent to cleave-SS—. The GSH cleavage reagent included: 1 mol/L Tris-Base, 0.5 mol/L sodium chloride, 0.05% (v/v) Tween 20, and GSH, at a pH of 9.0. Four parallel tests were set with GSH concentrations of 10 mmol/L, 20 mmol/L, 30 mmol/L, and 50 mmol/L, respectively. Then, the surface of the solid carrier was further irradiated with excitation light from a 532 nm laser and a 640 nm laser separately, and optical signals were acquired to obtain image 2. The fluorescence signals of the two images were counted, and the cleavage efficiency was analyzed. The cleavage percentage of —SS—=(the number of optical signal points before cleavage−the number of optical signal points after cleavage)/the number of optical signal points before cleavage×100%; alternatively, the residual percentage of the disulfide bonds on the surface of the solid carrier was calculated, where the residual percentage of the disulfide bonds=the number of optical signal points after cleavage/the number of optical signal points before cleavage×100%. The results are shown in FIG. 2. The results showed that the GSH cleavage reagent exhibited good cleavage effect on —SS—, and the cleavage effect was better in the concentration range of 20 mmol/L-50 mmol/L.

### Example 3

Test for efficiency of azidomethoxy cleavage by THPP, including the following steps:

Nucleotide solutions of dA-N3-Atto647N and dC-N3-Atto532 were prepared, respectively, and 9°N enzyme was added to each nucleotide solution to form extension reagents. The extension reagent containing dA-N3-Atto647N and 9°N enzyme was named as dA-N3-Atto647N extension reagent, and the extension reagent containing dC-N3-Atto532 and 9°N enzyme was named as dC-N3-Atto532 extension reagent. In the extension reagents, the concentration of the 9°N enzyme was set to 0.02 mg/mL; and the concentrations of dA-N3-Atto647N and dC-N3-Atto532 were 50 nmol/L and 500 nmol/L, respectively.

A synthesis reagent was introduced onto the surface of the solid carrier at a temperature of 37° C. for reaction for 2 min. After the reaction was completed, the surface of the solid carrier was washed.

The surface of the solid carrier was separately irradiated with excitation light from a 532 nm laser and a 640 nm laser and the fluorophores on the extension reagents were excited to generate optical signals, and the fluorescence signals of the solid carrier were acquired to obtain image 1. Subsequently, reaction was allowed at 37° C. for 2 min using a THPP cleavage reagent to cleave azidomethoxy. The THPP cleavage reagent included: 1 mol/L Tris-Base, 0.5 mol/L sodium chloride, 0.05% Tween 20, and THPP, at a pH of 9.0. Parallel tests were set with THPP concentrations of 10 mmol/L, 25 mmol/L, 50 mmol/L, and 100 mmol/L, respectively. Then, the surface of the solid carrier was further irradiated with excitation light from a 532 nm laser and a 640 nm laser separately, and optical signals were acquired to obtain image 2. The fluorescence signals of the two images were counted, and the cleavage efficiency was analyzed.

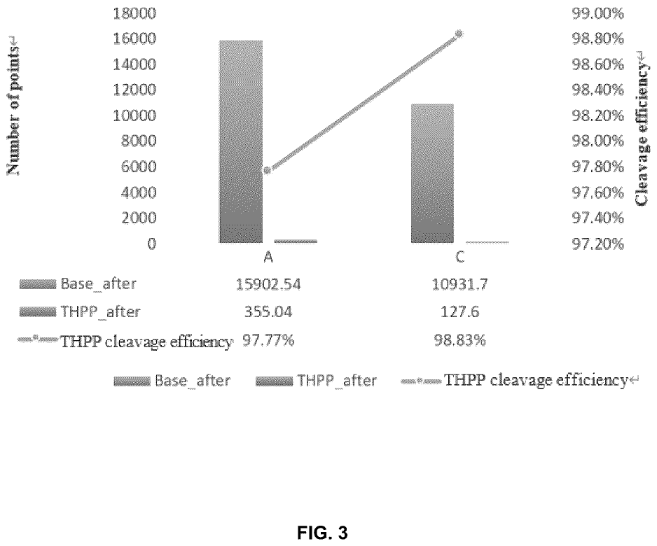

The cleavage percentage of azidomethoxy=(the number of optical signal points before cleavage−the number of optical signal points after cleavage)/the number of optical signal points before cleavage×100%; alternatively, the residual percentage of azidomethoxy=the number of optical signal points after cleavage/the number of optical signal points before cleavage×100%. The results showed that THPP had good cleavage effect on azidomethoxy. The cleavage effect when the concentration of THPP was 10 mmol/L is shown in FIG. 3.

### Example 4

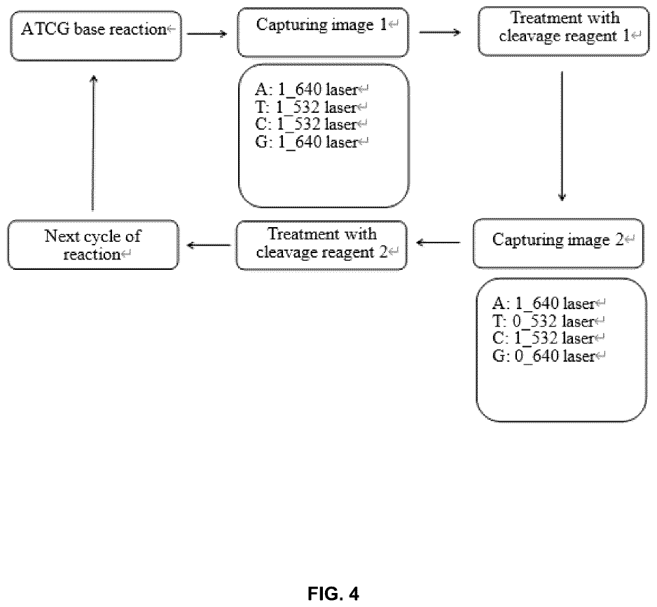

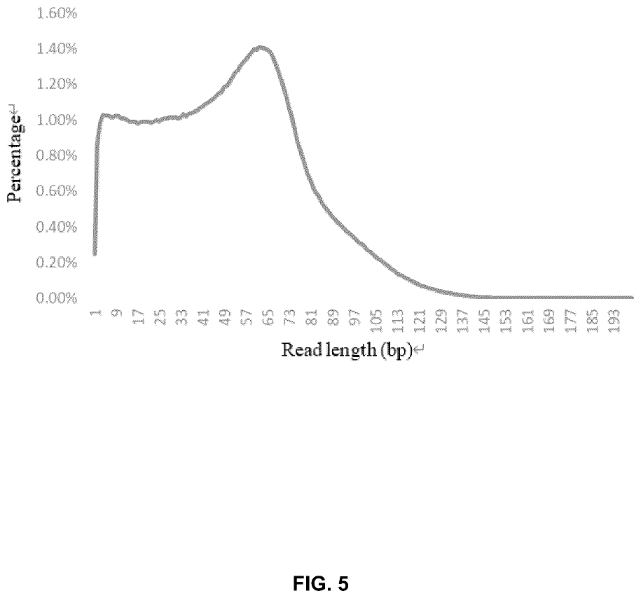

A two-color sequencing method is provided, including the following steps:
after rehydration of the chip, hybridizing a gDNA library; preparing base solutions of dA-N3-Atto647N (50 nmol/L), dT-N3-SS-Atto532 (50 nmol/L), dC-N3-Alexa Fluor532 (500 nmol/L), and dG-N3-SS-CY5 (5 nmol/L), with 0.01 mg/mL 9°N polymerase, and a buffer including: 50 mmol/L Tris-Base at a pH of 8.8, 50 mmol/L NaCl, 10 mmol/L (NH₄)₂SO₄, and 3 mmol/L MgSO₄;
providing:
cleavage reagent 1 (GSH cleavage reagent): 1 mol/L Tris-Base, 0.5 mol/L sodium chloride, 50 mmol/L GSH, and 0.05% Tween 20, at a pH of 9.0;
cleavage reagent 2 (THPP cleavage reagent): 1 mol/L Tris-Base, 0.5 mol/L sodium chloride, 10 mmol/L THPP, and 0.05% Tween 20, at a pH of 9.0;
the two-color dual-cleavage sequencing process is shown in FIG. 4;
the specific steps are as follows:
(1) providing a surface-processed solid carrier including a plurality of sites, each of the sites having a non-amplified immobilized nucleic acid template-primer complex (single-molecule nucleic acid template);
(2) preparing nucleotide solutions of dA-N3-Atto647N at a concentration of 50 nmol/Lol/L, dT-N3-SS-Atto532 at a concentration of 50 nmol/Lol/L, dC-N3-Alexa Fluor532 at a concentration of 500 nmol/L, and dG-N3-SS-CY5 at a concentration of 5 nmol/Lol/L (buffer: 50 mmol/L Tris-Base, at a pH of 8.8; 50 mmol/L NaCl; 10 mmol/L (NH₄)₂SO₄; and 3 mmol/L MgSO₄), and adding 9°N enzyme to a concentration of 0.01 mg/mL to form an extension reagent;
(3) adding the extension reagent to the surface of the solid carrier, utilizing excitation light from a 532 nm laser and a 640 nm laser to separately irradiate the surface of the solid carrier for 60 ms, imaging the solid carrier, and capturing image 1;
(4) preparing a GSH cleavage reagent: 1 mol/L Tris-Base, 0.5 mol/L sodium chloride, 50 mmol/L GSH, and 0.05% Tween 20, at a pH of 9.0; adding the cleavage reagent to the surface of the solid carrier to perform a cleavage reaction on the disulfide bonds;
(5) utilizing excitation light from a 532 nm laser and a 640 nm laser to separately irradiate the surface of the solid carrier for 80 ms, imaging the solid carrier, and capturing image 2;
(6) preparing a THPP cleavage reagent: 1 mol/L Tris-Base, 0.5 mol/L sodium chloride, 10 mmol/L THPP, and 0.05% Tween 20, at a pH of 9.0; adding the cleavage reagent to the surface of the solid carrier, and performing a cleavage reaction on the azide group; and
(7) repeating the above steps (2)-(7) 50 times, i.e., performing sequencing for 50 cycles. The read length distribution of the 50 cycles is shown in FIG. 5. As can be seen from the figure, the main peak of the read length distribution of the 50-cycle sequencing was around 50 bp, and the proportion of sequencing termination before 45 cycles was relatively low.

Two groups of parallel experiments were performed on two identical sequencing platforms, and the sequencing throughput, read length, and mean error rate of the 50 cycles were statistically analyzed. The results are shown in Table 3 below, where the throughput included the number of Reads uniquely aligned to the genome in the 1024×1024 pixel region in the center of 400 fields of view, i.e., FOVs, the error rate (error rate=mismatch rate+deletion-type error rate+insertion-type error rate) was less than 8.6%, indicating good stability.

TABLE 3

| Throughput | Read | Mismatch | Deletion-type | Insertion-type | Error | Density |
|---|---|---|---|---|---|---|
| (400 FOV) | length | rate | error rate | error rate | rate | (reads/μm²) |
| 4693152 | 40.750 | 2.83% | 2.85% | 2.66% | 8.34% | 3.35 |
| 4586528 | 40.219 | 2.57% | 3.09% | 2.35% | 8.01% | 3.15 |
| 4694032 | 40.519 | 2.58% | 3.12% | 2.38% | 8.08% | 3.13 |
| 4403928 | 40.495 | 2.75% | 3.27% | 2.53% | 8.55% | 3.31 |

What is claimed is:

1. A two-color sequencing method, comprising the following steps:
causing four types of reversible terminators to undergo a polymerization reaction with a nucleic acid template-primer complex, wherein the four types of reversible terminators are each independently linked to a nucleotide or a derivative thereof and a fluorophore via a cleavable group, the cleavable groups of two types of the reversible terminators being identical, the fluorophores of two types of the reversible terminators being identical, and the cleavable groups carrying identical fluorophores being different or requiring different cleavage conditions;
exciting the fluorophores to emit fluorescence, and acquiring fluorescence signals to obtain a first image;
cleaving one type of the cleavable groups by a first reaction;
acquiring fluorescence signals of the remaining fluorophores to obtain a second image; and
determining, based on the first image and the second image, types of reversible terminators participating in the polymerization reaction.

2. The two-color sequencing method according to claim 1, wherein the four types of reversible terminators are respectively a first reversible terminator, a second reversible terminator, a third reversible terminator, and a fourth reversible terminator, and the four types of reversible terminators each independently comprise: a nucleotide or a derivative thereof, a fluorophore, and a first cleavable group and/or a second cleavable group linking the nucleotide or the derivative thereof and the fluorophore, the nucleotide or the derivative thereof being selected from adenine or a derivative thereof, thymine or a derivative thereof, cytosine or a derivative thereof, or guanine or a derivative thereof, and the fluorophore being selected from a first fluorophore and a second fluorophore that are distinguishable in color;
image 1 comprises a first signal, a second signal, a third signal, and a fourth signal from the first reversible terminator, the second reversible terminator, the third reversible terminator, and the fourth reversible terminator, respectively, the first signal and the second signal being different from the third signal and the fourth signal;
image 2 comprises a fifth signal, a sixth signal, a seventh signal, and an eighth signal from the first reversible terminator, the second reversible terminator, the third reversible terminator, and the fourth reversible terminator, respectively, the sixth signal being different from the second signal and the seventh signal being different from the third signal; and
after acquiring the second image or determining the types of reversible terminators participating in the polymerization reaction, the other type of the cleavable groups is cleaved by a second reaction.

3. The two-color sequencing method according to claim 2, wherein cleavability of the second cleavable group is stronger than cleavability of the first cleavable group, and the first reaction cleaves the second cleavable group, and
the second reaction cleaves the first cleavable group.

4. The two-color sequencing method according to claim 3, wherein
the first cleavable group is selected from a group comprising at least one of —N3-, —CH₂F, —C(O)NHBu, —CHF₂, —C(O)OH, —C₆, and/or —ArC₆.

5. The two-color sequencing method according to claim 4, wherein the first cleavable group is selected from at least one of —N3-, —CH₂F, —C(O)NHBu, —CHF₂, —C(O)OH, —C₆, and/or —ArC₆.

6. The two-color sequencing method according to claim 2, wherein the first fluorophore and the second fluorophore are each independently selected from one of or a combination of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin.

7. The two-color sequencing method according to claim 6, wherein a color combination of the first fluorophore and the second fluorophore is one of the following combinations:
the fluorescence color of the first fluorophore being red, and the fluorescence color of the second fluorophore being green;
the fluorescence color of the first fluorophore being red, and the fluorescence color of the second fluorophore being blue;
the fluorescence color of the first fluorophore being green, and the fluorescence color of the second fluorophore being red;
the fluorescence color of the first fluorophore being green, and the fluorescence color of the second fluorophore being blue;
the fluorescence color of the first fluorophore being blue, and the fluorescence color of the second fluorophore being red; and
the fluorescence color of the first fluorophore being blue, and the fluorescence color of the second fluorophore being green.

8. The two-color sequencing method according to claim 7, wherein the fluorophore having a fluorescence color of green comprises Atto532, Alexa Fluor532, Atto Rho6G, or CY3;
the fluorophore having a fluorescence color of red comprises: Atto647N, CY5, GM656, or Alexa Fluor647; and
the fluorophore having a fluorescence color of blue comprises: Atto390, Alexa Fluor405, or coumarin.

9. The two-color sequencing method according to claim 2, wherein the first signal is the same as the second signal, and the third signal is the same as the fourth signal.

10. The two-color sequencing method according to claim 9, wherein any two types of the reversible terminators have the same cleavable group.

11. The two-color sequencing method according to claim 10, wherein the fluorophores of the reversible terminators having the same cleavable group have different fluorescence colors.

12. The two-color sequencing method according to claim 11, wherein the reversible terminators have the following structures:
the structure of the first reversible terminator being: a first nucleotide or a derivative thereof-the first cleavable group-the first fluorophore;
the structure of the second reversible terminator being: a second nucleotide or a derivative thereof-the second cleavable group-the first fluorophore;
the structure of the third reversible terminator being: a third nucleotide or a derivative thereof-the second cleavable group-the second fluorophore; and
the structure of the fourth reversible terminator being: a fourth nucleotide or a derivative thereof-the first cleavable group-the second fluorophore,
wherein the first nucleotide or the derivative thereof, the second nucleotide or the derivative thereof, the third nucleotide or the derivative thereof, and the fourth nucleotide or the derivative thereof are independently selected from one of adenine or a derivative thereof, thymine or a derivative thereof, cytosine or a derivative thereof, and guanine or a derivative thereof, and the first nucleotide or the derivative thereof, the second nucleotide or the derivative thereof, the third nucleotide or the derivative thereof, and the fourth nucleotide or the derivative thereof are different from one another.

13. The two-color sequencing method according to claim 12, wherein the four types of reversible terminators comprise at least one of the following combinations 1-16; or the four types of reversible terminators comprise at least one of the following combinations 17-32:
combination 1: adenine or a derivative thereof-N3-Atto647N; thymine or a derivative thereof-SS-Atto532; cytosine or a derivative thereof-N3-Alexa Fluor532; and guanine or a derivative thereof-SS-CY5;
combination 2: adenine or a derivative thereof-N3-Atto647N; thymine or a derivative thereof-SS-Atto532; cytosine or a derivative thereof-N3-Alexa Fluor532; and guanine or a derivative thereof-SS-Atto647N;
combination 3: adenine or a derivative thereof-N3-Atto647N; thymine or a derivative thereof-SS-Alexa Fluor532; cytosine or a derivative thereof-N3-Atto532; and guanine or a derivative thereof-SS-Atto647N;
combination 4: adenine or a derivative thereof-N3-CY5; thymine or a derivative thereof-SS-Atto532; cytosine or a derivative thereof-N3-Alexa Fluor532; and guanine or a derivative thereof-SS-CY5;
combination 5: adenine or a derivative thereof-N3-CY5; thymine or a derivative thereof-SS-Alexa Fluor532; cytosine or a derivative thereof-N3-Atto532; and guanine or a derivative thereof-SS-CY5;
combination 6: adenine or a derivative thereof-N3-CY5; thymine or a derivative thereof-SS-Atto532; cytosine or a derivative thereof-N3-Atto532; and guanine or a derivative thereof-SS-CY5;
combination 7: adenine or a derivative thereof-N3-CY5; thymine or a derivative thereof-SS-Alexa Fluor532; cytosine or a derivative thereof-N3-Alexa Fluor532; and guanine or a derivative thereof-SS-CY5;
combination 8: adenine or a derivative thereof-N3-Atto647N; thymine or a derivative thereof-SS-Atto532; cytosine or a derivative thereof-N3-Atto532; and guanine or a derivative thereof-SS-CY5;
combination 9: adenine or a derivative thereof-N3-Atto647N; thymine or a derivative thereof-SS-Alexa Fluor532; cytosine or a derivative thereof-N3-Atto532; and guanine or a derivative thereof-SS-CY5;
combination 10: adenine or a derivative thereof-N3-Atto647N; thymine or a derivative thereof-SS-Alexa Fluor532; cytosine or a derivative thereof-N3-Alexa Fluor532; and guanine or a derivative thereof-SS-CY5;
combination 11: adenine or a derivative thereof-N3-CY5; thymine or a derivative thereof-SS-Alexa Fluor532; cytosine or a derivative thereof-N3-Alexa Fluor532; and guanine or a derivative thereof-SS-Atto647N;
combination 12: adenine or a derivative thereof-N3-CY5; thymine or a derivative thereof-SS-Atto532; cytosine or a derivative thereof-N3-Atto532; and guanine or a derivative thereof-SS-Atto647N;
combination 13: adenine or a derivative thereof-N3-CY5; thymine or a derivative thereof-SS-Atto532; cytosine or a derivative thereof-N3-Alexa Fluor532; and guanine or a derivative thereof-SS-Atto647N;
combination 14: adenine or a derivative thereof-N3-CY5; thymine or a derivative thereof-SS-Alexa Fluor532; cytosine or a derivative thereof-N3-Atto532; and guanine or a derivative thereof-SS-Atto647N;
combination 15: adenine or a derivative thereof-N3-Atto647N; thymine or a derivative thereof-SS-Alexa Fluor532; cytosine or a derivative thereof-N3-Alexa Fluor532; and guanine or a derivative thereof-SS-Atto647N;
combination 16: adenine or a derivative thereof-N3-Atto647N; thymine or a derivative thereof-SS-Atto532; cytosine or a derivative thereof-N3-Atto532; and guanine or a derivative thereof-SS-Atto647N;
combination 17: adenine or a derivative thereof-N3-Atto532; thymine or a derivative thereof-SS-Atto647N; cytosine or a derivative thereof-N3-Atto647N; and guanine or a derivative thereof-SS-Atto532;
combination 18: adenine or a derivative thereof-N3-Alexa Fluor532; thymine or a derivative thereof-SS-Atto647N; cytosine or a derivative thereof-N3-Atto647N; and guanine or a derivative thereof-SS-Atto532;
combination 19: adenine or a derivative thereof-N3-Atto532; thymine or a derivative thereof-SS-Atto647N; cytosine or a derivative thereof-N3-Atto647N; and guanine or a derivative thereof-SS-Alexa Fluor532;
combination 20: adenine or a derivative thereof-N3-Alexa Fluor532; thymine or a derivative thereof-SS-Atto647N; cytosine or a derivative thereof-N3-Atto647N; and guanine or a derivative thereof-SS-Alexa Fluor532;
combination 21: adenine or a derivative thereof-N3-Atto532; thymine or a derivative thereof-SS-CY5; cytosine or a derivative thereof-N3-Atto647N; and guanine or a derivative thereof-SS-Atto532;
combination 22: adenine or a derivative thereof-N3-Alexa Fluor532; thymine or a derivative thereof-SS-CY5; cytosine or a derivative thereof-N3-Atto647N; and guanine or a derivative thereof-SS-Atto532;
combination 23: adenine or a derivative thereof-N3-Atto532; thymine or a derivative thereof-SS-CY5; cytosine or a derivative thereof-N3-Atto647N; and guanine or a derivative thereof-SS-Alexa Fluor532;
combination 24: adenine or a derivative thereof-N3-Alexa Fluor532; thymine or a derivative thereof-SS-CY5; cytosine or a derivative thereof-N3-Atto647N; and guanine or a derivative thereof-SS-Alexa Fluor532;
combination 25: adenine or a derivative thereof-N3-Atto532; thymine or a derivative thereof-SS-Atto647N; cytosine or a derivative thereof-N3-CY5; and guanine or a derivative thereof-SS-Atto532;
combination 26: adenine or a derivative thereof-N3-Alexa Fluor532; thymine or a derivative thereof-SS-Atto647N; cytosine or a derivative thereof-N3-CY5; and guanine or a derivative thereof-SS-Atto532;
combination 27: adenine or a derivative thereof-N3-Atto532; thymine or a derivative thereof-SS-Atto647N; cytosine or a derivative thereof-N3-CY5; and guanine or a derivative thereof-SS-Alexa Fluor532;
combination 28: adenine or a derivative thereof-N3-Alexa Fluor532; thymine or a derivative thereof-SS-Atto647N; cytosine or a derivative thereof-N3-CY5; and guanine or a derivative thereof-SS-Alexa Fluor532;
combination 29: adenine or a derivative thereof-N3-Alexa Fluor532; thymine or a derivative thereof-SS-CY5; cytosine or a derivative thereof-N3-CY5; and guanine or a derivative thereof-SS-Atto532;
combination 30: adenine or a derivative thereof-N3-Atto532; thymine or a derivative thereof-SS-CY5; cytosine or a derivative thereof-N3-CY5; and guanine or a derivative thereof-SS-Alexa Fluor532;
combination 31: adenine or a derivative thereof-N3-Atto532; thymine or a derivative thereof-SS-CY5; cytosine or a derivative thereof-N3-CY5; and guanine or a derivative thereof-SS-Atto532; and
combination 32: adenine or a derivative thereof-N3-Alexa Fluor532; thymine or a derivative thereof-SS-CY5; cytosine or a derivative thereof-N3-CY5; and guanine or a derivative thereof-SS-Alexa Fluor532.

14. The two-color sequencing method according to claim 13, wherein in combinations 1-32, —N3- linked to adenine or a derivative thereof is replaced with —SS—, —N3- linked to cytosine or a derivative thereof is replaced with —SS—, —SS— linked to thymine or a derivative thereof is replaced with —N3-, and —SS— linked to guanine or a derivative thereof is replaced with —N3-; or
in combinations 1-32, —N3- linked to adenine or a derivative thereof is replaced with —SS—, and —SS— linked to guanine or a derivative thereof is replaced with —N3-; or
in combinations 1-32, —SS— linked to thymine or a derivative thereof is replaced with —N3-, and —N3- linked to cytosine or a derivative thereof is replaced with —SS—.

15. The two-color sequencing method according to claim 2, wherein the second reversible terminator and the third reversible terminator comprise the first cleavable group, and the first cleavable group links the nucleotide or the derivative thereof and the second cleavable group.

16. The two-color sequencing method according to claim 15, wherein the reversible terminators have the following structures:
the structure of the first reversible terminator being: a first nucleotide or a derivative thereof-the first cleavable group-the first fluorophore;
the structure of the second reversible terminator being: a second nucleotide or a derivative thereof-the first cleavable group-the second cleavable group-the first fluorophore;
the structure of the third reversible terminator being: a third nucleotide or a derivative thereof-the first cleavable group-the second cleavable group-the second fluorophore;
the structure of the fourth reversible terminator being: a fourth nucleotide or a derivative thereof-the first cleavable group-the second fluorophore,
wherein the first nucleotide or the derivative thereof, the second nucleotide or the derivative thereof, the third nucleotide or the derivative thereof, and the fourth nucleotide or the derivative thereof are independently selected from one of adenine or a derivative thereof, thymine or a derivative thereof, cytosine guanine or a derivative thereof, and guanine or a derivative thereof, and the first nucleotide or the derivative thereof, the second nucleotide or the derivative thereof, the third nucleotide or the derivative thereof, and the fourth nucleotide or the derivative thereof are different from one another.

17. The two-color sequencing method according to claim 16, wherein the four types of reversible terminators comprise at least one of the following combinations 33-48; or the four types of reversible terminators comprise at least one of the following combinations 49-64:
combination 33: adenine or a derivative thereof-N3-Atto647N; thymine or a derivative thereof-N3-SS-Atto532; cytosine or a derivative thereof-N3-Alexa Fluor532; and guanine or a derivative thereof-N3-SS-CY5;
combination 34: adenine or a derivative thereof-N3-Atto647N; thymine or a derivative thereof-N3-SS-Atto532; cytosine or a derivative thereof-N3-Alexa Fluor532; and guanine or a derivative thereof-N3-SS-Atto647N;
combination 35: adenine or a derivative thereof-N3-Atto647N; thymine or a derivative thereof-N3-SS-Alexa Fluor532; cytosine or a derivative thereof-N3-Atto532; and guanine or a derivative thereof-N3-SS-Atto647N;
combination 36: adenine or a derivative thereof-N3-CY5; thymine or a derivative thereof-N3-SS-Atto532; cytosine or a derivative thereof-N3-Alexa Fluor532; and guanine or a derivative thereof-N3-SS-CY5;
combination 37: adenine or a derivative thereof-N3-CY5; thymine or a derivative thereof-N3-SS-Alexa Fluor532; cytosine or a derivative thereof-N3-Atto532; and guanine or a derivative thereof-N3-SS-CY5;
combination 38: adenine or a derivative thereof-N3-CY5; thymine or a derivative thereof-N3-SS-Atto532; cytosine or a derivative thereof-N3-Atto532; and guanine or a derivative thereof-N3-SS-CY5;
combination 39: adenine or a derivative thereof-N3-CY5; thymine or a derivative thereof-N3-SS-Alexa Fluor532; cytosine or a derivative thereof-N3-Alexa Fluor532; and guanine or a derivative thereof-N3-SS-CY5;
combination 40: adenine or a derivative thereof-N3-Atto647N; thymine or a derivative thereof-N3-SS-Atto532; cytosine or a derivative thereof-N3-Atto532; and guanine or a derivative thereof-N3-SS-CY5;
combination 41: adenine or a derivative thereof-N3-Atto647N; thymine or a derivative thereof-N3-SS-Alexa Fluor532; cytosine or a derivative thereof-N3-Atto532; and guanine or a derivative thereof-N3-SS-CY5;
combination 42: adenine or a derivative thereof-N3-Atto647N; thymine or a derivative thereof-N3-SS-Alexa Fluor532; cytosine or a derivative thereof-N3-Alexa Fluor532; and
guanine or a derivative thereof-N3-SS-CY5;
combination 43: adenine or a derivative thereof-N3-CY5; thymine or a derivative thereof-N3-SS-Alexa Fluor532; cytosine or a derivative thereof-N3-Alexa Fluor532; and guanine or a derivative thereof-N3-SS-Atto647N;
combination 44: adenine or a derivative thereof-N3-CY5; thymine or a derivative thereof-N3-SS-Atto532; cytosine or a derivative thereof-N3-Atto532; and guanine or a derivative thereof-N3-SS-Atto647N;
combination 45: adenine or a derivative thereof-N3-CY5; thymine or a derivative thereof-N3-SS-Atto532; cytosine or a derivative thereof-N3-Alexa Fluor532; and guanine or a derivative thereof-N3-SS-Atto647N;
combination 46: adenine or a derivative thereof-N3-CY5; thymine or a derivative thereof-N3-SS-Alexa Fluor532; cytosine or a derivative thereof-N3-Atto532; and guanine or a derivative thereof-N3-SS-Atto647N;
combination 47: adenine or a derivative thereof-N3-Atto647N; thymine or a derivative thereof-N3-SS-Alexa Fluor532; cytosine or a derivative thereof-N3-Alexa Fluor532; and guanine or a derivative thereof-N3-SS-Atto647N;
combination 48: adenine or a derivative thereof-N3-Atto647N; thymine or a derivative thereof-N3-SS-Atto532; cytosine or a derivative thereof-N3-Atto532; and guanine or a derivative thereof-N3-SS-Atto647N;
combination 49: adenine or a derivative thereof-N3-Atto532; thymine or a derivative thereof-N3-SS-Atto647N; cytosine or a derivative thereof-N3-Atto647N; and guanine or a derivative thereof-N3-SS-Atto532;
combination 50: adenine or a derivative thereof-N3-Alexa Fluor532; thymine or a derivative thereof-N3-SS-Atto647N; cytosine or a derivative thereof-N3-Atto647N; and guanine or a derivative thereof-N3-SS-Atto532;
combination 51: adenine or a derivative thereof-N3-Atto532; thymine or a derivative thereof-N3-SS-Atto647N; cytosine or a derivative thereof-N3-Atto647N; and guanine or a derivative thereof-N3-SS-Alexa Fluor532;
combination 52: adenine or a derivative thereof-N3-Alexa Fluor532; thymine or a derivative thereof-N3-SS-Atto647N; cytosine or a derivative thereof-N3-Atto647N; and guanine or a derivative thereof-N3-SS-Alexa Fluor532;
combination 53: adenine or a derivative thereof-N3-Atto532; thymine or a derivative thereof-N3-SS-CY5; cytosine or a derivative thereof-N3-Atto647N; and guanine or a derivative thereof-N3-SS-Atto532;
combination 54: adenine or a derivative thereof-N3-Alexa Fluor532; thymine or a derivative thereof-N3-SS-CY5; cytosine or a derivative thereof-N3-Atto647N; and guanine or a derivative thereof-N3-SS-Atto532;
combination 55: adenine or a derivative thereof-N3-Atto532; thymine or a derivative thereof-N3-SS-CY5; cytosine or a derivative thereof-N3-Atto647N; and guanine or a derivative thereof-N3-SS-Alexa Fluor532;
combination 56: adenine or a derivative thereof-N3-Alexa Fluor532; thymine or a derivative thereof-N3-SS-CY5; cytosine or a derivative thereof-N3-Atto647N; and guanine or a derivative thereof-N3-SS-Alexa Fluor532;
combination 57: adenine or a derivative thereof-N3-Atto532; thymine or a derivative thereof-N3-SS-Atto647N; cytosine or a derivative thereof-N3-CY5; and guanine or a derivative thereof-N3-SS-Atto532;
combination 58: adenine or a derivative thereof-N3-Alexa Fluor532; thymine or a derivative thereof-N3-SS-Atto647N; cytosine or a derivative thereof-N3-CY5; and guanine or a derivative thereof-N3-SS-Atto532;
combination 59: adenine or a derivative thereof-N3-Atto532; thymine or a derivative thereof-N3-SS-Atto647N; cytosine or a derivative thereof-N3-CY5; and guanine or a derivative thereof-N3-SS-Alexa Fluor532;
combination 60: adenine or a derivative thereof-N3-Alexa Fluor532; thymine or a derivative thereof-N3-SS-Atto647N; cytosine or a derivative thereof-N3-CY5; and guanine or a derivative thereof-N3-SS-Alexa Fluor532;
combination 61: adenine or a derivative thereof-N3-Alexa Fluor532; thymine or a derivative thereof-N3-SS-CY5; cytosine or a derivative thereof-N3-CY5; and guanine or a derivative thereof-N3-SS-Atto532;
combination 62: adenine or a derivative thereof-N3-Atto532; thymine or a derivative thereof-N3-SS-CY5; cytosine or a derivative thereof-N3-CY5; and guanine or a derivative thereof-N3-SS-Alexa Fluor532;
combination 63: adenine or a derivative thereof-N3-Atto532; thymine or a derivative thereof-N3-SS-CY5; cytosine or a derivative thereof-N3-CY5; and guanine or a derivative thereof-N3-SS-Atto532; and
combination 64: adenine or a derivative thereof-N3-Alexa Fluor532; thymine or a derivative thereof-N3-SS-CY5; cytosine or a derivative thereof-N3-CY5; and guanine or a derivative thereof-N3-SS-Alexa Fluor532.

18. The two-color sequencing method according to claim 17, wherein in combinations 33-64, a single cleavable group-N3- linked to adenine or a derivative thereof is replaced with a double cleavable group-N3-SS—, a single cleavable group-N3- linked to cytosine or a derivative thereof is replaced with —N3-SS—, a double cleavable group-N3-SS— linked to thymine or a derivative thereof is replaced with a single cleavable group-N3-, and a double cleavable group-N3-SS— linked to guanine or a derivative thereof is replaced with a single cleavable group-N3-; or
in combinations 33-64, a single cleavable group-N3- linked to adenine or a derivative thereof is replaced with a double cleavable group-N3-SS—, and a double cleavable group-N3-SS— linked to guanine or a derivative thereof is replaced with a single cleavable group-N3-; or
in combinations 33-64, a double cleavable group-N3-SS— linked to thymine or a derivative thereof is replaced with a single cleavable group-N3-, and a single cleavable group-N3- linked to cytosine or a derivative thereof is replaced with a double cleavable group-N3-SS—.

19. The two-color sequencing method according to claim 13, wherein
the reversible terminators are combination 1; and
in combination 1, the working concentrations of the reversible terminators are: adenine or the derivative thereof-N3-Atto647N at 10 nmol/L-700 nmol/L; thymine or the derivative thereof-N3-SS-Atto532 at 5 nmol/L-60 nmol/L; cytosine or the derivative thereof-N3-Alexa Fluor532 at 10 nmol/L-700 nmol/L; and guanine or the derivative thereof-N3-SS-CY5 at 0.05 nmol/L-5 nmol/L.

20. The two-color sequencing method according to claim 19, wherein
a cleavage reagent for cleaving the first cleavable group is an organic phosphine cleavage reagent; and
a cleavage reagent for cleaving the second cleavable group is a reducing agent.
